(19) United States
(12) Patent Application Publication
(10) Pub. No.: US 2026/0216297 A1
(43) Pub. Date: Jul. 30, 2026

(54) STABILIZED FORMULATIONS OF INSULIN AND INSULIN ANALOGS

(71) Applicant: Portal Diabetes Inc., Westfield, IN (US)

(72) Inventor: Nicolas Mauricio Jeset Varas-Molina, Westfield, IN (US)

(21) Appl. No.: 19/373,003

(22) Filed: Oct. 29, 2025

Related U.S. Application Data
(60) Provisional application No. 63/713,492, filed on Oct. 29, 2024.

(51) Int. Cl. A61K 38/28; A61K 47/02; A61K 47/10; A61K 47/22; A61K 47/34

(52) U.S. Cl. CPC A61K 38/28; A61K 47/02; A61K 47/10; A61K 47/22; A61K 47/34

(57) ABSTRACT

A pharmaceutical formulation presented, comprising an insulin or an insulin analog, and at least one protective excipient, in high concentration, selected from: a protective osmolyte, a surfactant, and a phenolic preservative. In embodiments, the insulin or insulin analog is one or more of: regular human insulin, insulin lispro, insulin aspart, insulin glulisine, insulin glargine, insulin detemir, or insulin degludec. In some embodiments, at least one of: the concentration of the protective osmolyte confers an osmolarity greater than 250 mOsm/L, the concentration of the surfactant is greater than 0.1% w/v, or the concentration of the phenolic preservative is greater than 0.4% w/v. In some embodiments, insulin API is dissolved and incubated for 20 to 40 minutes at high pH before the protective excipients are added.

### CROSS REFERENCE TO RELATED APPLICATIONS

This application claims the benefit of U.S. Provisional Patent Application No. 63/713,492, filed on Oct. 29, 2024 and entitled “STABILIZED FORMULATIONS OF INSULIN AND INSULIN ANALOGS,” the entire disclosure of which is hereby incorporated herein by reference.

### TECHNICAL FIELD

This invention relates to pharmaceutical compositions and methods for formulating human insulin or insulin analogs that enhance physical and chemical stability. In particular, this invention relates to using a stabilizing formulation process coupled with optimized concentrations of stabilizing excipients to increase the physical and chemical stability of human insulin or its analogs. More particularly, this invention relates to the use of a high pH pre-formulation treatment coupled with an optimized concentration of surfactants, phenolic preservatives, and protective osmolytes alone or in combination with each other to improve the physical and chemical stability of formulated insulin.

### BACKGROUND OF THE INVENTION

Insulin has been used for over 100 years to control blood glucose levels in people with diabetes. Insulin has been a driving force for innovation in protein production, purification, engineering, formulation, delivery, and pharmacology (Jarosinski et al., 2021). Different analogs of native human insulin have been formulated to modify its pharmacokinetics and to better emulate its physiological release (Jarosinski et al., 2022). However, insulin and its analogs have an intrinsic propensity to aggregate into amyloidogenic fibrils (Brange et al., 1997). Such fibrillation leads to loss of drug potency, potential immunogenicity, and clogging of catheters in insulin pumps (Woods et al., 2012). This aggregation is accelerated by the presence of “seeds,” or proto-fibrils, which are a natural byproduct of the insulin manufacturing process. Once fibrils begin forming, their growth is exponential. It is thought that fibrils accrete insulin molecules from both ends, then break once they reach a critical size. This creates twice the number of ends from which to capture additional insulin molecules. Reaching a critical mass of seeds takes time, however. The time before the exponential process of fibril formation takes off is called the fibrillation lag time and is the primary measure of the physical stability of insulin formulations.

Further, insulin and its analogs are prone to chemical degradation. For example, insulin is susceptible to chemical modifications such as, for example, deamidations, isomerization, and redox modifications, which first occur in susceptible amino acids such as, for example, Asn, Gin, and Cys. Insulin is also susceptible to covalent modifications that generate dimers and other high molecular weight products (HMWPs) (Brange & Langkjoer, 1993). HMWPs can be immunogenic and are of particular concern to regulators.

Both aggregation and chemical degradation can increase exponentially as temperatures and motion increase (Brange et al., 1997; Huus et al., 2006). This intrinsic instability is partially mitigated in current pharmaceutical formulations by use of stabilizing additives such as zinc ions and phenolic ligands that promote insulin's native, reversable hexameric aggregation structure (Jarosinski et al., 2021; Qafary et al., 2022). Nonetheless, despite these strategies, due to the susceptibility of conventional insulin formulations to degradation at, and especially above, room temperature, drug regulatory agencies have required refrigeration for the storage of insulin formulations. Thus, most insulin formulations must be discarded (a) if left unrefrigerated for more than 28 days (with some exceptions that can last for up to 8 weeks) or (b) if left inside a body-worn pump for more than 48-72 hours (Heinemann et al., 2021). These requirements place a significant constraint on the global distribution and access to insulin formulations, especially in places where maintaining a cold chain up and into the patient's home is difficult or impossible (Bhutta et al., 2021; Devi, 2021). Pens, vials, and pump cartridges that must be carried with the patient, especially in places or seasons when temperatures exceed “room temperature,” must often be discarded unused, leading to waste and increased costs. Insulin's intrinsic instability also places constraints on the development of new analogs (such as, for example, glucose-responsive insulins) or new delivery technologies like implantable pumps that could be built to hold enough insulin for several months of use at body temperature (37° C.) if an insulin formulation that was stable enough could be found.

What is needed are solutions to these and other problems.

### SUMMARY OF THE INVENTION

Pharmaceutical compositions and formulation methods are presented that focus on the synergistic effects of combining the protective impact of a pre-formulation high pH treatment of an insulin active pharmaceutical agent (“API”), with the use of optimized concentrations of stabilizing excipients, specifically surfactants, phenolic preservatives, and protective osmolytes, to enhance the physical and chemical stability of formulated human insulin or its analogs.

In embodiments, a high pH pre-treatment includes solubilizing an insulin API, generally in a purified crystalline or powder form, and using a solution comprised of a base (e.g., sodium or potassium hydroxide) at a sufficient concentration that causes the pH after dissolution to be between 9 and 12. In embodiments, this treatment may be maintained for between 20 and 40 minutes, inclusive, in order to increase physical stability but not increase chemical degradation.

In embodiments, the high pH pre-treatment can be synergistically combined with one or more of the following stabilizing excipients at optimized concentrations:
(1) A surfactant, at a concentration above 0.1% w/v but below the critical micelle concentration of that surfactant. (In contrast, concentrations conventionally used in insulin formulation are around 0.001% w/v). In one embodiment, Poloxamer surfactants such as, for example, Poloxamer 188 or Poloxamer 407, may be used. In other embodiments, for example, Polysorbates (20 or 80), sugar-based surfactants, Polyethylene glycol-based surfactants, amphiphilic acrylamide copolymers, Brijs, or any other surfactant, may be used.
(2) A phenolic preservative at a concentration above 0.4% w/v (>0.15% is required to inhibit microbial growth). In one embodiment this can be, for example, a concentration of between 0.50% and 0.60% w/v. In embodiments, the phenolic preservative may include phenol, metacresol, or a combination of both. Alternatively, other embodiments may include, for example, other phenolic preservatives such as parabens (methylparaben, propylparaben, etc.), phenoxyethanol, benzyl alcohol, or any combination thereof.
(3) A protective osmotic agent (i.e., not glycerol or inorganic salts) at a concentration selected to achieve isotonicity with physiologic fluids such as blood, extracellular fluids, or peritoneal fluid (e.g., around 250-350 mOsm/L). In one embodiment Proline or Trehalose may be used as protective osmotic agents. In other embodiments, protective osmolytes such as, for example, trimethylamine N-oxide (TMAO), glycine, taurine, hypotaurine, arginine, serine, ectoine, betaine, sarcosine, sucrose, sorbitol, mannitol, glycerophosphocholine (GPC), dimethylsulfoniopropionate (DMSP), etc. may be used.

In addition, a method is presented for evaluating the resistance of a protein formulation to amyloid fibril growth seeded or accelerated by interaction with a hydrophobic surface. Finally, pharmaceutical formulations developed or selected by use of that method are presented.

### BRIEF DESCRIPTION OF THE DRAWINGS

FIG. 1 depicts an example rotary mixer with vials oriented radially, according to an embodiment.

FIG. 2 illustrates surfactant optimization: fibrillation lag time for different concentrations of Poloxamer 188, according to an embodiment.

FIG. 3 illustrates preservative optimization: fibrillation lag time for different concentrations of phenol, according to an embodiment.

FIG. 4 illustrates osmolyte impact on Desamido A21 at 25° C. for four different osmolytes, according to an embodiment.

FIG. 5 illustrates osmolyte impact on Desamido A21 at 37° C. for four different osmolytes, according to an embodiment.

FIG. 6 illustrates osmolyte impact on other related substances at 25° C. for four different osmolytes, according to an embodiment.

FIG. 7 illustrates osmolyte impact on other related substances at 37° C. for four different osmolytes, according to an embodiment.

FIG. 8 illustrates osmolyte impact on High Molecular Weight Proteins (“HMWP”) A21 at 25° C. for four different osmolytes, according to an embodiment.

FIG. 9 illustrates osmolyte impact on HMWP A21 at 37° C. for four different osmolytes, according to an embodiment.

FIG. 10 illustrates physical stability after pre-treatment: fibrillation lag times for different 50 mM NaOH pretreatment durations, according to an embodiment.

FIG. 11 illustrates Desamido A21 percentage after pre-treatment for different 50 mM NaOH pretreatment incubation times, according to an embodiment.

FIG. 12 illustrates other related substances: percentage after pre-treatment for different 50 mMol NaOH pretreatment incubation times, according to an embodiment.

FIG. 13 illustrates HMWP percentage after pre-treatment for different 50 mM NaOH pretreatment incubation times (plot W-01=0 minutes; plot W-02=10 minutes; plot W-03=20 minutes; plot W-04=30 minutes; plot W-05=40 minutes; and plot W-06=50 minutes), according to an embodiment.

FIG. 14 illustrates other related substances chromatograms: from pre-treatment study for the longest two 50 mM NaOH pretreatment incubation times (plot W-01=0 minutes; plot W-02=10 minutes; plot W-03=20 minutes; plot W-04=30 minutes; plot W-05=40 minutes; and plot W-06=50 minutes), according to an embodiment.

FIG. 15 illustrates other related substances chromatograms from a pre-treatment study for different 50 mM NaOH pretreatment incubation times (plot W-05=40 minutes; and plot W-06=50 minutes), according to an embodiment.

FIG. 16 illustrates HMWP chromatograms from a pre-treatment study for various 50 mM NaOH pretreatment durations, according to an embodiment.

FIG. 17 illustrates HMWP chromatograms from a pre-treatment study for two different 50 mM NaOH pretreatment incubation times (shown in plots W-05 and W-06), according to an embodiment.

FIG. 18 illustrates PI-U500 fibrillation lag time on plate reader/nephelometer running at 300 RPM and 37° C., as compared with Unsuman (a U500 version of Insuman Implantable U400), according to an embodiment.

FIG. 19 illustrates PI-U500 fibrillation lag time on a rotary mixer running at 80 RPM and 37° C., as compared with Unsuman, according to an embodiment.

FIG. 20 illustrates PI-U500 HMWP accumulation at 5° C. for PI U-500, as compared with Unsuman, according to an embodiment.

FIG. 21 illustrates PI-U500 HMWP accumulation at 30° C. for PI U-500, as compared with Unsuman, according to an embodiment.

FIG. 22 illustrates PI-U500 HMWP accumulation at 37° C. for PI U-500, as compared with Unsuman, according to an embodiment.

FIG. 23 illustrates PI-U500 HMWP accumulation at 50° C. for PI U-500, as compared with Unsuman, according to an embodiment.

FIG. 24 illustrates PI-U500 Desamido A21 accumulation at 5° C. for PI U-500, as compared with Unsuman, according to an embodiment.

FIG. 25 illustrates PI-U500 Desamido A21 accumulation at 30° C. for PI U-500, as compared with Unsuman, according to an embodiment.

FIG. 26 illustrates PI-U500 High Desamido A21 accumulation at 37° C. for PI U-500, as compared with Unsuman, according to an embodiment.

FIG. 27 illustrates PI-U500 Desamido A21 accumulation at 50° C. for PI U-500, as compared with Unsuman, according to an embodiment.

FIG. 28 illustrates PI-U500 other related substances accumulation at 5° C. for PI U-500, as compared with Unsuman, according to an embodiment.

FIG. 29 illustrates PI-U500 other related substances accumulation at 30° C. for PI U-500, as compared with Unsuman, according to an embodiment.

FIG. 30 illustrates PI-U500 other related substances accumulation at 37° C. for PI U-500, as compared with Unsuman, according to an embodiment.

FIG. 31 illustrates PI-U500 other related substances accumulation at 50° C. for PI U-500, as compared with Unsuman, according to an embodiment.

FIG. 32 illustrates pharmacokinetics (upper plot) and glucodynamics (lower plot) in male beagle dogs after intraperitoneal injection: PI-U500 vs Unsuman (as noted, a U500 version of Insuman Implantable U400), according to an embodiment.

FIG. 33 illustrates insulin Lispro formulations, according to an embodiment.

FIG. 34 illustrates insulin Lispro formulations fibrillation lag times on a rotary mixer running at 80 RPM and 37° C., according to an embodiment.

FIG. 35 illustrates insulin Lispro formulations fibrillation lag times on a nephelometer plate running at 300 RPM and 37° C., according to an embodiment.

FIG. 36 illustrates insulin Aspart formulations, according to an embodiment.

FIG. 37 illustrates insulin Aspart formulations fibrillation lag times on a rotary mixer running at 80 RPM and 37° C., according to an embodiment.

FIG. 38 illustrates insulin Aspart formulations fibrillation lag times on a nephelometer plate running at 300 RPM and 37° C., according to an embodiment.

FIG. 39 illustrates insulin Glargine formulations, according to an embodiment.

FIG. 40 illustrates insulin Glargine formulations fibrillation lag times on a rotary mixer running at 80 RPM and 37° C., according to an embodiment.

FIG. 41 illustrates insulin Glargine formulations fibrillation lag times on a nephelometer plate running at 300 RPM and 37° C., according to an embodiment.

### DETAILED DESCRIPTION

This disclosure relates to pharmaceutical compositions and methods for formulating human insulin or insulin analogs that enhance physical and chemical stability. In particular, the disclosure relates to a stabilizing formulation process coupled with optimized concentrations of stabilizing excipients to increase the physical and chemical stability of human insulin or its analogs. More particularly, this disclosure relates to the use of a high pH pre-formulation treatment coupled with an optimized concentration of surfactants, phenolic preservatives, and protective osmolytes—either alone or in combination with each other—to improve the physical and chemical stability of formulated insulin. Therefore, the disclosure further relates to methods for treating diabetes using the stabilized insulin formulations.

As noted above, commercially available formulations of human insulin or its analogs are designed to be stable at refrigerated conditions but have not been optimized to resist prolonged exposures at or above room temperature. This minimally acceptable stability has been achieved by promoting insulin's hexameric structure through the addition of zinc (Zn) ions, which recapitulate the naturally occurring Zn-insulin hexamer structures found inside the pancreas. Serendipitously, the phenolic preservatives (phenol, meta-cresol, etc.) used in insulin formulations further stabilize the insulin hexamer into a more stable form known as the “R6 hexamer.” Phenolic preservative concentrations in commercial formulations have generally been optimized only to inhibit microorganism growth, as shown in Table A below:

TABLE A

| | Year | | Concentration |
|---|---|---|---|
| Insulin / Insulin Analog | Introduced | Preservative | (mg/ml) |
| Humulin R (Regular) | 1982 | Metacresol | 2.5 |
| Humulin N (NPH) | 1982 | Metacresol + | 1.6 + 0.65 |
| | | phenol | |
| Humalog (Insulin Lispro) | 1996 | Metacresol | 3.15 |
| Novolog (Insulin Aspart) | 2000 | Metacresol + | 1.72 + 1.5 |
| | | phenol | |
| Novolin R (Regular) | 1988 | Metacresol | 3.0 |
| Lantus (Insulin Glargine) | 2000 | Metacresol | 2.7 |
| Levemir (Insulin Detemir) | 2005 | Metacresol + | 2.06 + 1.8 |
| | | phenol | |
| Tresiba (Insulin Degludec) | 2012 | Metacresol + | 1.72 + 1.5 |
| | | phenol | |
| Apidra (Insulin Glulisine) | 2004 | Metacresol | 3.15 |

The discovery that slightly higher concentrations of preservative (3.15 vs 2.5 mg/ml—0.315 vs 0.25% w/v) stabilize the R6 hexamer of insulin lispro was made by Bruce Frank when developing the Humalog formulation. However, this level of preservative has not been universally applied to subsequent insulin analog formulations because the benefits of this slightly higher concentration did not apply to all insulin analogs. As noted, stability at elevated temperatures has not been a focus of previous insulin formulation development.

There have been efforts to develop novel excipients that can improve insulin's resistance to physical aggregation (fibrillation) by supramolecular pegylation with cucurbit [7] uril/polyethylene glycol (Webber et al., 2016), by either utilizing a “biochaperone” (Heise et al., 2019), or by utilizing acrylamide carrier/dopant copolymers (Mann et al., 2020). Although somewhat effective in delaying aggregation, these technologies have proven insufficient, since they (1) only result in modest delays in aggregation; (2) do not remove fibrillogenic seeds or pre-fibrils generated during insulin manufacturing; (3) do not protect against chemical degradation; (4) have unknown effects on insulin pharmacokinetics; and (5) introduce excipients not previously used in approved pharmaceutical formulations which have unknown toxicities.

The standard formulation process involves dissolving an insulin API in acid (pH ~2), which is well below human insulin's iso-electric point of around 5.3 (where the protein's net charge is zero and the protein is the least soluble; analogs of human insulin each have their own iso-electric points). The formulation is generally then adjusted to physiological pH by adding NaOH. It has also been claimed in PCT patent application WO2004096266A1 to Poulsen, et al. that the physical stability of insulin formulations may be improved by alkaline rather than acidic dissolution. Poulsen claimed that dissolving insulin at a pH between 8 and 12 and holding it there for between 5 and 20 minutes at a temperature between 0° C. and 15° C. would increase the physical stability of an insulin formulation (as measured by fibrillation lag time) compared to standard formulation methods. However, Poulsen only provided data on a few pH levels and a 15-minute duration, as shown in Table B:

TABLE B
Experimental Examples in Poulsen

| Experiment | 1 | | 2 | | | |
|---|---|---|---|---|---|---|
| Insulin | Human | | Aspart | | | |
| Formulation | NPH | | Novolog | | | |
| Stress type | Rotation @37° C. | | Shaking @ 37° C. | | | |
| Batch | 1 | 2 | 1 | 2 | 3 | 4 |
| Replicates | 25 | 25 | 5 | 5 | 5 | 5 |
| Dissolution pH | 11.3 | 2 | 7.9 | 9.6 | 10.4 | 2.2 |
| Time at | 15 | 15 | Not | Not | Not | Not |
| pH (min) | | | Dis- | Dis- | Dis- | Dis- |
| | | | closed | closed | closed | closed |
| Relative | 2.3 | 1 | 1.4 | 1.2 | 1.5 | 1 |
| lag time | | | | | | |

Poulson did not provide any guidance or insight as to optimal ranges of pH, time, or order of component addition, or suggest any synergies with excipients in the formulation. Further, it is also known that prolonged exposure to elevated pH stimulates chemical degradation and the formation of HMWPs. As is known, an increase in physical stability alone is not sufficient to allow for a thermostable insulin formulation and does not provide protection against chemical degradation.

What is therefore needed are improved thermostable insulin formulations that are more resistant to both physical and chemical degradation so that they can be stored or used at or above room temperature for prolonged periods of time. Technologies that are both transferable among the various current or future insulin analogs, and that are also compatible with varying methods of delivery (vials/syringes, pens, pumps, etc.), are desired. Such technologies would simplify the cold chain or even render it obsolete without affecting the stability or potency of the drug. Various embodiments, as described below, present such improved thermostable formulations that protect against both fibrillation and chemical degradation.

The inventors have discovered that various synergistic techniques may improve insulin formulation stability at higher temperatures. In embodiments, such formulations may include: (i) a process that can remove most, if not all, of the pre-fibril “seeds” in an insulin API that are known to promote the aggregation/fibrillation of insulin; (ii) an excipient, usually a surfactant, that prevents the insulin from interacting with hydrophobic surfaces (air, oils, plastics, etc.), which surfaces can promote fibril seed formation; (iii) a phenolic preservative at higher concentrations than currently found in commercial insulin formulations that can further stabilize insulin hexamers at elevated temperatures; and (iv) a protective osmotic agent selected to reduce chemical degradation, as opposed to conventionally used osmotic agents such as glycerol or inorganic salts.

As noted above, in embodiments, an insulin API may be, for example, (a) treated with high pH, and then combined with one or more of: (b) a surfactant in high concentration, (c) a phenolic preservative, and (d) a protective osmotic agent. Each of these elements is next described in detail. It is noted that as used herein the term “insulin” may, unless specified otherwise, refer to any type of insulin, including insulin analogs.

A. Initial Treatment of Insulin API with High pH

It is known that insulin aggregates can be broken apart by high pH. Further, high pH can chemically degrade the insulin molecule and can also degrade other excipients in a given formulation. Thus, it is impractical to utilize a formulation that is maintained at high pH. Nonetheless, in embodiments, a high-pH treatment of the insulin API may be performed that, when applied for a short period of time, permanently increases insulin stability without chemically degrading the insulin molecule. This process is believed to break apart any existing seed or pre-fibril that may have been generated during the production, purification, storage, or transport of the insulin API, thereby delaying the stochastic process of seed generation that is believed to be a prerequisite for the exponential growth of amyloid fibrils and increasing the physical stability of any insulin formulation made with API treated with this process. In embodiments, utilizing such a high pH pre-treatment adds physical stability that is synergistic to physical stability enhancements provided by high concentrations of phenolic preservatives, protective osmolytes, and surfactants. In embodiments, this high pH treatment may be done before adding some or all of the excipients in a formulation, as certain excipients may interfere with action of the pre-treatment. For example, it was found that adding all of the excipients before the high pH treatment reduced the stability of the ultimate formulation as compared to adding all of the excipients after the high pH treatment had been terminated. In some embodiments, all excipients may be added at once, in a pre-made buffer, right after the high pH treatment and neutralization.

In embodiments, the API may be incubated at between pH 10 and 11 for between 20 and 40 minutes (see Example No. 5, described below) before adjusting the pH to between 3.8 and 7.8. The incubation pH can be anywhere in that range. In some embodiments, the API may be incubated in the high pH for 30 minutes. In embodiments, the final pH depends on the isoelectric point of the insulin analog. For example, most insulin analogs are formulated at pH 7.0 to 7.8. However, insulin glargine must be formulated at approximately pH 4 because it is insoluble at pH 7.

### B. Surfactants in High Concentration

The interaction of insulin with hydrophobic surfaces causes a structural rearrangement of the insulin molecule that exposes its hydrophobic core. This facilitates hydrophobic interactions between side chains (intra-protein and inter-protein interactions) that further enhance a β-sheet-rich structure (a characteristic of amyloid-like fibrils) that can “seed” the formation and subsequent exponential growth of fibrils. In embodiments, high concentrations of surfactants may be used to effectively prevent these interactions by coating the hydrophobic surfaces. In such embodiments, one side of the surfactant molecule faces the hydrophobic surface, leaving a hydrophilic face and creating a now hydrophilic surface that is protein-friendly. The most hydrophobic surface that insulin molecules can be exposed to is air, which is present as headspace and/or bubbles inside all insulin containers (such as, for example, vials, syringes, pens, cartridges, pump reservoirs, etc.). However, hydrophobic oils, for example, silicone oil, can also be present in these devices, such as, for example, as lubricants for plunger seals. Moreover, agitation due to body movement when a user carries a pen or an insulin pump increases the portion of the formulation exposed to the air/water interface or other hydrophobic surface, accelerating the seed formation process. Thus, coating these surfaces is of particular value in containers that are moved around or otherwise agitated, such as pump reservoirs.

In embodiments, surfactants can coat these air bubbles or other hydrophobic surfaces and reduce this seed formation. Some insulin products have been approved that contain surfactants, such as, for example, Insuman Implantable U400 containing 0.01 mg/ml of Poloxamer 171 (approved to market in Europe), or Lantus, a glargine insulin formulation with a worldwide distribution, which contains 0.02 mg/ml of Polysorbate 20. However, these uses of surfactants have been restricted to such low concentrations. This is likely due to the conventional assumption that high hydrophobicity surfactants or surfactant concentrations can affect protein structure.

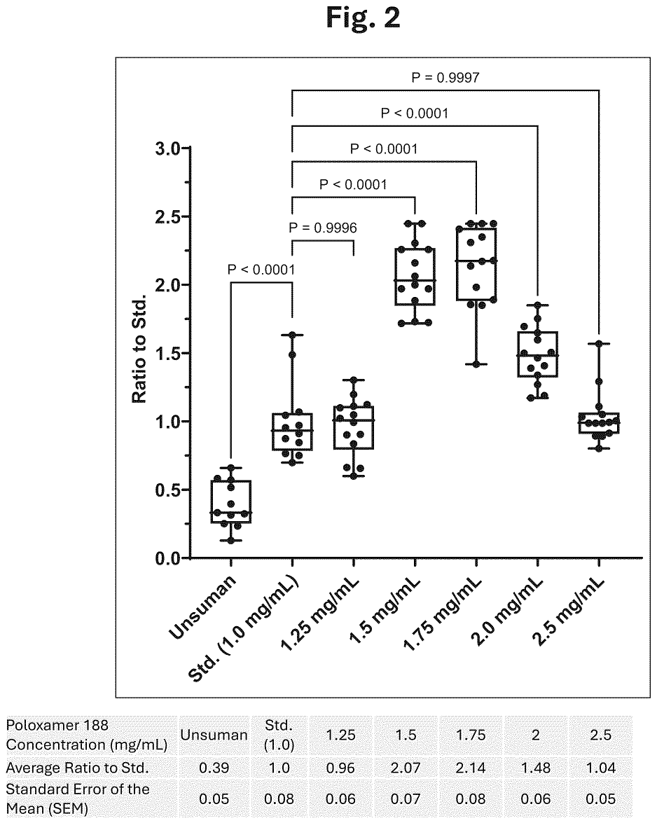

In embodiments, substantially higher concentrations of surfactants (e.g., 1-2 mg/ml) significantly increase stability, particularly for prolonged exposure at elevated temperatures, so long as (1) the surfactant's critical micelle concentration (CMC) is not reached and (2) surfactant-related hydrophobicity is not high enough to interact with the protein and destabilize. In embodiments, the upper limit for surfactant concentration may be determined by making formulations with elevated concentrations and testing them for: (1) micelle formation—for example by using dynamic light scattering, surface tension monitoring, or turbidity methods, and (2) for adverse effects on protein stability such as physical or chemical degradation. As an example, with reference to FIG. 2, at 2.5 mg/ml concentration of Poloxamer 188, the CMC is starting to be approached, as the effectiveness decreases. Thus, one can assume that if the concentration of the surfactant here would be further increased, a detrimental effect would be observed.

In embodiments, the protective effect from surfactants is synergistic with the other stabilization mechanisms disclosed herein. As an

In embodiments, one surfactant may be used. However, in some embodiments, combinations of surfactants may also be used, for example if experiments suggest that the optimum Hydrophilic-Lipophilic Balance (HLB) number for the surfactant used in a formulation may be between that of two available surfactants.

Selection of one surfactant over another is often a matter of trial and error. Some surfactants show better compatibility with a particular protein than do other surfactants. For example, in embodiments, Poloxamer 188 or Poloxamer 407 are better for insulin formulations because of their lower hydrophobicity index (HLB number) compared to other surfactants, including other Poloxamers. It is believed that more hydrophobic surfactants may interfere with insulin hexamer formation and therefore adversely affect its physical stability. Thus, experiments were run with surfactants with at least 70% hydrophilic content. For example, Poloxamer 188 has 80% of hydrophilic molecules while Poloxamer 407 has 70%. By contrast, Poloxamers 171 and 181 have only 10% hydrophilic content, as illustrated in Table C below.

TABLE C
Characteristics of Poloxamers

| | Molecular Weight | Percentage of |
|---|---|---|
| | of Hydrophobic | Hydrophilic |
| Poloxamer | core | Content |
| 171 | 17 | 10% |
| 181 | 18 | 10% |
| 188 | 18 | 80% |
| 407 | 40 | 70% |

As noted above, in embodiments, exemplary formulations include a surfactant at a concentration greater than 0.1% w/v (as opposed to conventional insulin formulations which only use surfactants at ~0.001% w/v). The surfactant's purpose is to coat external hydrophobic surfaces like air or plastic with its own hydrophobic side while interacting with the protein (insulin) through its hydrophilic side. Therefore, in embodiments, by using a significantly higher concentration of surfactants (for example, at greater than 100× of conventional concentrations) larger hydrophobic surfaces are covered and dynamic hydrophobic surfaces are more quickly recovered. This decreases the likelihood of the insulin interacting with such hydrophobic surfaces, and thus increases the physical stability of the insulin.

Accordingly, in embodiments, much higher concentrations (e.g., 0.15 to 0.2 w/v %) of surfactant are used than conventional formulations, but always remaining below the CMC and the protein-destabilization level. In embodiments, this is the point where surfactant effectiveness decreases, as it starts to interact with itself, as opposed to coating the hydrophobic surfaces.

### C. Phenolic Preservatives

In embodiments, increasing the concentration of preservative to above 0.50% w/v (vs. 0.315% w/v maximum in conventional formulations) further improves stability at elevated temperatures. This is likely by better stabilizing R6 hexamers at temperatures where the equilibrium between hexamers and monomers would otherwise shift more toward the unstable monomers. In embodiments, such an increase in concentration also reduces the risk that the preservative's concentration in the formulation might fall below a required antimicrobial concentration due to evaporation of the preservative through container components.

In embodiments, the high pH pre-treatment and high surfactant levels can be synergistically combined with phenolic preservatives at a concentration above 0.4% w/v (>0.15% is required to inhibit microbial growth). In one embodiment this can be, for example, a concentration of between 0.50% and 0.60% w/v. In embodiments, the phenolic preservative may be phenol, metacresol, or a combination of both where the combined concentration is above 0.4% w/v.

Alternatively, other embodiments may include, for example, phenolic preservatives such as parabens (methylparaben, propylparaben, etc.), phenoxyethanol, benzyl alcohol, or any combination thereof. In some embodiments, the high concentration phenolic preservative may be implemented alone, without one of, or either, high pH treatment and high surfactant level.

### D. Protective Osmolytes

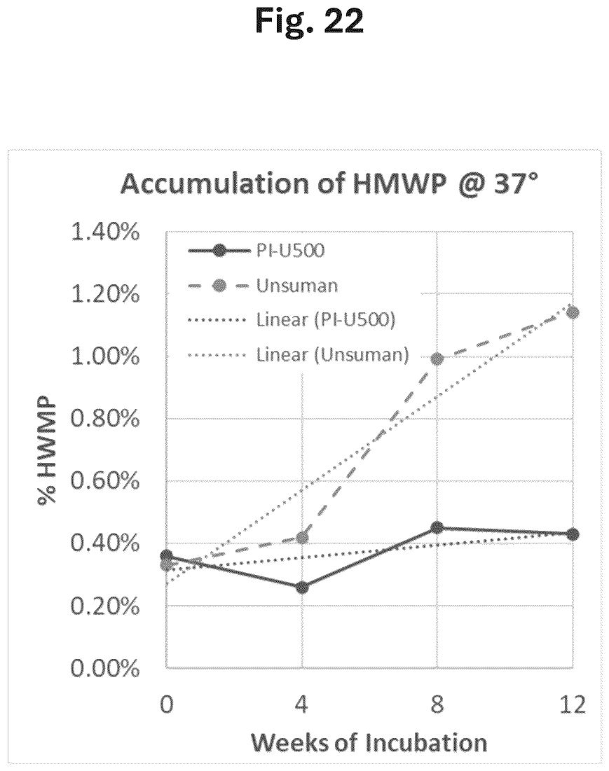

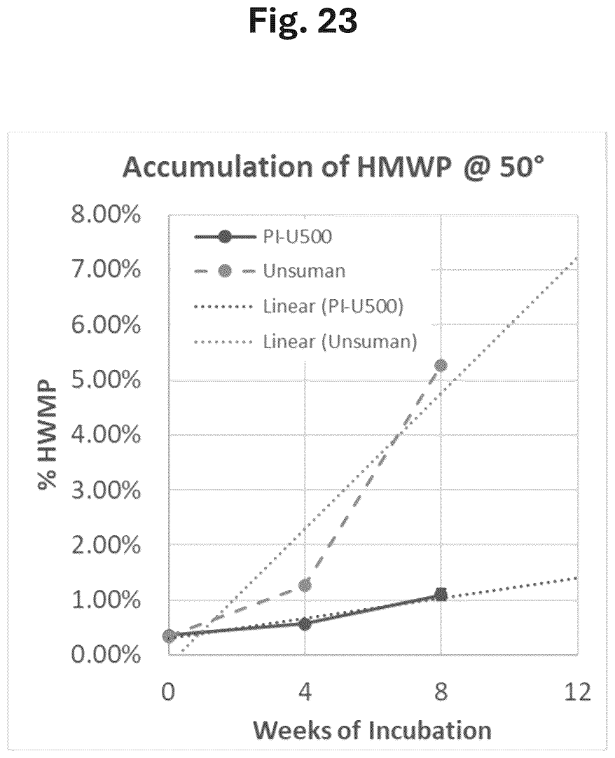

Osmolytes are known as protein-protecting agents, especially for protection from thermal degradation. Protective osmolytes are naturally occurring small molecules and have been employed in molecular biology research to optimize PCR (polymerase chain reaction) or to optimize proteomic protocols (Pepelnjak et al., 2024). While the mechanism by which protective osmolytes stabilize proteins is unclear, protective osmolyte performance has been shown to be protein specific. Some protective osmolytes, most notably trehalose, have been used in drug formulations to protect drugs in lyophilization and freeze-thaw cycles. However, no osmolyte has been used to protect against temperature-induced degradation at or above room temperature for any protein drug formulation, including insulin. In embodiments, a properly selected protective osmolyte, such as, for example, proline or trehalose, can significantly improve the chemical stability of an insulin formulation. Surprisingly, proline has a dramatic impact on the formation of HMWP by human insulin, greatly reducing its rate of appearance at high temperatures when compared with other osmotic agents—as shown in FIGS. 22 and 23. This is a size of protective effect never before reported, or known to be used, for an osmolyte. It was found that L-Proline had a significant effect on a particular degradation product, HMWP, and was far superior to other osmolytes in this regard. HMWP happens to be the degradation product of greatest concern for insulin.

To be effective, protective osmolytes must have high concentrations (1 mol/L has been the standard in molecular biology research) to interact favorably with proteins. However, insulin drug products, and most parenteral products, are designed to be isosmotic with respect to physiological fluids (250-350 mOsm/L) to prevent local irritation and pain at the administration site. Thus, it was discovered that to make protective osmolyte concentration high enough to affect chemical stability without exceeding the osmolarity desired for the product (which can be iso-, hypo-, or hyper-osmolar in different embodiments), the protective osmolytes must be the only osmotic agents in the formulation (e.g., no glycerol or inorganic salts). In embodiments, eliminating glycerol has the added benefit of avoiding an excipient that is susceptible to high variability in quality between manufacturing batches. It was further discovered that, by using such a protective osmolyte along with high concentrations of phenolics and surfactants while minimizing salts in a formulation, chemical degradation may be reduced without increasing physical degradation.

Some protective osmolytes are not advised, such as TMAO, which is toxic, or sucrose, because it degrades insulin.

Given the above discussion, in embodiments, compositions and methods for formulating human insulin or its analogs to enhance their physical and chemical stability are provided, that allow for prolonged in-use and shelf stability at or above room temperature. In embodiments, these formulations may be used to treat diabetes by any method of delivery, including, for example nasal, oral, intravenous, subcutaneous, or intraperitoneal, via syringes, pens, patches, or pumps.

An advantage of various embodiments that the disclosed techniques are applicable to multiple insulin analogs, both those in current use, as well as those yet to be deployed or discovered. All insulin analogs, both current and future-developed, will have similar degradation pathways because they all have similar protein sequence and structure. As a result, formulations of all such analogs may be stabilized using the techniques disclosed herein.

Insulin analogs and insulin analog formulations have been designed to create rapid-acting, ultra-rapid acting, long-acting, ultra-long acting, or biphasic drugs. All of them, as well as future analogs, can benefit from being formulated for prolonged stability at or above room temperature, in accordance with various embodiments. The extent to which their respective stabilities may be prolonged depends on the insulin analog, but for currently available analogs (lispro, aspart, glulisine, glargine, detemir and degludec) at least a two-fold increase in the in-use life can be achieved. Some embodiments hereof target rapid-acting analogs which are currently labeled to last for 2-3 days inside of a cartridge that is inserted into a body-worn subcutaneous pump. Formulations containing the same insulin analogs (e.g., lispro, aspart, and glulisine) can be stabilized, according to various embodiments, to remain stable for 6 or more days at in-use temperatures. Other embodiments target high-concentration insulins where the stability may be increased from 1.5 months to at least 3 months, and even as much as six, nine, or even more months of in-use stability at room (30° C.) or body temperature (37° C.). Techniques according to embodiments of the present invention can be applied to insulin formulations in the range of U20 to U2000 (20 to 2000 international units/ml).

As noted, various embodiments combine (a) a pre-formulation high pH treatment of insulin API that increases physical stability with optimized concentrations of at least one stabilizing excipient selected from the following: (b) surfactant, (c) phenolic preservative, and (d) protective osmolyte. Some embodiments may comprise one, two or more classes of these stabilizing excipients without first performing the high pH treatment of the API. Such embodiments, it is noted, do not exclude other excipients that may be needed to optimize pharmacological properties or further stabilize insulin. In particular, any of the embodiments described herein may, for example, include the use of Zinc in any form (oxide, chloride, sulfate, etc.) to obtain basal levels of stability, or may, for example, implement the complete removal of Zinc (such as, for example, by using chelators such as EDTA or the like) to accelerate hexamer disassembly after injection and speed the onset of action. In embodiments, some of the stabilizing excipients may be added before the high pH treatment and some after the completion of the treatment. In other embodiments, excipients meant to accelerate subcutaneous absorption of insulin, such as, for example, treprostinil, sodium citrate, Nicotinamide or L-arginine, can be combined with the stabilizing excipients herein or the high-pH treatment.

In embodiments, pH buffers may optionally be used to stabilize the pH at any desired level. Such buffers may include, for example, Tromethamine (Tris), phosphate, citrate, histidine, and the like. In embodiments, buffering will depend on the type of insulin formulation. Thus, for example, for a U500 insulin, no buffer is advised. For more diluted insulins, buffering will depend on the desired pH, or potential applications. Therefore, in embodiments, a preferred choice will be API and formulation specific.

### Method for Discovery

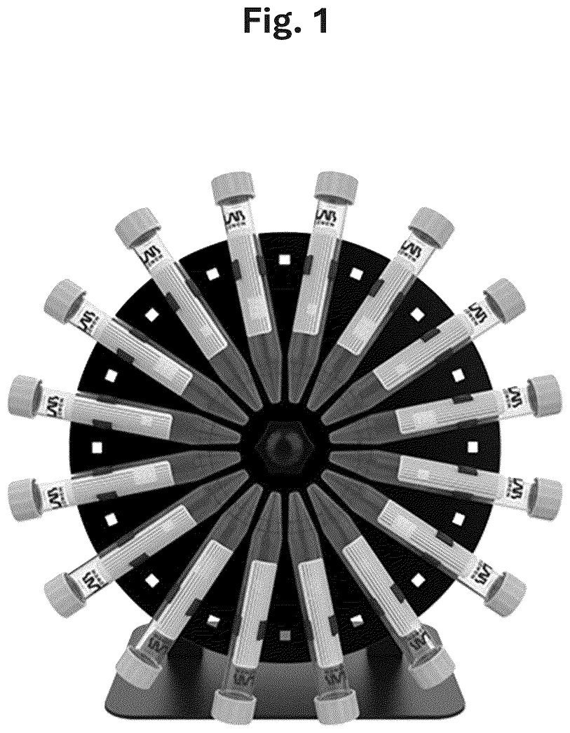

The present disclosure further includes a method for evaluating the physical stability of a candidate formulation. While plate nephelometry to determine the fibrillation lag time of a protein formulation may be used, there has not to date been disclosed a method for evaluating the resistance of a formulation to fibrillation due to the formulation's interaction with a hydrophobic surface. In embodiments, sealed vials or other containers containing less than 100% of their total volume of a formulation are agitated in a rolling or rotatory fashion to stimulate dynamic coating by the formulation of a hydrophobic surface. In some embodiments, the vials are placed on a roller mixer. In other embodiments, the rolling or rotary agitation is achieved by attaching the containers to a rotary mixer, such as, for example, the one shown in FIG. 1. For example, the containers might be attached so that they are oriented radially or circumferentially. In embodiments, the containers might hold volumes of 0.1, 0.5, 1.0, 2.0, 5.0, 10.00, 50.0, 100, or 200 mL, for example. The rotary wheel may be oriented so that it is within a vertical plane with respect to gravity, or at an angle off of the vertical plane (such as, for example, 10, 20. 30, 45, 60 or 89 degrees off of the vertical plane). In embodiments, the rotary mixer might turn at 10, 50, 100, or 300 rpm, for example. For example, the speed of the mixer may be determined based on the viscosity of the formulation such that the liquid formulation has time to flow from one end of the container to the other once or twice in each rotation, thus maximizing the exposure of the formulation to hydrophobic surfaces. In embodiments, the rotating mixer's speed may also be set so that the exposure of protein molecules in the formulation is long enough for them to unfold and expose any hydrophobic core they may have. Too brief an exposure (due to too rapid rotation) may allow for the proteins to “recover” from their surface interaction and may therefore reduce the proportion of surface-exposed protein molecules that fully unfold. In embodiments, the containers may be 10 to 90 percent or 25 to 75 percent full, for example. In embodiments, the remaining volume of the containers may be filled with a gas, a hydrophobic liquid, or a vacuum. An air/water interface is an extremely hydrophobic surface so repeated rolling past such a surface can be highly fibrillogenic.

In embodiments, the inner surface of the vial may have a hydrophobic coating. Such coatings may include oils, waxes, silicone, polytetrafluoroethelent, or other hydrophobic substances known, or that may later be known, in the art. In embodiments, the vials may also contain beads or particulates with hydrophobic surfaces.

In an alternative embodiment, a gas may be bubbled through the formulation.

In embodiments, the formulation is maintained at a fixed or variable temperature while it is being agitated. For example, the formulation may be maintained at a refrigerated (e.g., 2-8° C.) room (e.g., 25-30° C.), body (e.g., 37° C.), or stressing (e.g., 40-50° C.) temperatures, the latter obtained, for example, by placing the mixer in an incubator or waterbath. In embodiments, the formulation is monitored periodically (for example, once a minute, once an hour, or once, twice, or four times a day) for fibril formation. In embodiments, the monitoring may be done for example by light scattering (e.g., nephelometry), microscopy, spectroscopy, light obscuration, or ThT fluorescence. In embodiments, the physical stability of the formulation is then measured by determining the “lag time” between the start of the experiment and the detection of elevated levels of fibrils. In some embodiments, the onset of fibrillation may be determined by a significant increase in fibril measurement over the upper range of the lag phase. In other embodiments, the lag time may be determined by projecting backwards the linear growth phase of fibril levels.

A particular advantage of this approach is that it simulates, in some embodiments, on an accelerated basis, the fibrillogenic action of the movement of bubbles or air compartments within the reservoir of a body-worn or implantable pump and simulates the flow of a formulation past silicone oil on the surface of such a reservoir. This new approach simulates this real-world condition better than the conventional plate-reader nephelometer approach.

In another embodiment, this disclosure is directed to pharmaceutical formulations discovered or selected by utilizing this method of discovery. For example, candidate formulations may be evaluated for physical stability utilizing this method, and an optimal formulation selected, in whole or in part, based on the formulation's performance under this method.

Various embodiments hereof include formulations designed to be administered by any route. Such routes may include subcutaneous, intravenous, nasal, or intraperitoneal. In embodiments, any delivery method is also included, such as, for example, syringes, pens, patches, inhalers, or pumps to treat any type of Diabetes Mellitus or other high blood sugar condition.

### EXAMPLES

The following examples illustrate experiments performed to illustrate numerous aspects of various embodiments.

### Example No. 1—Methods for Evaluating Stability

To optimize formulations, the stability of each of was evaluated under up to three different stability methods done at room temperature or above.

The first method evaluates the resistance to physical stress caused by beads rapidly hitting the walls of a plastic plate that contains the insulin formulation. This is done using a 96-well plate containing 150 μl of insulin formulation and a Teflon bead. The plate is placed on a plate reader capable of measuring scattered light (nephelometry) or ThT fluorescence and also capable of constant agitation at or above 300 RPM and of heating the plate to 37° C. The signal is measured every 5-15 minutes; a 3-fold increase in turbidity or fluorescence over baseline indicates the start of the exponential fibril growth.

The second stability testing method also measures resistance to physical stress, but primarily that caused by air/water interface. In this test, a 2 ml sealed vial containing no more than 25% of the total volume of insulin formulation is agitated in a rolling or rotatory fashion to stimulate dynamic coating by insulin of the hydrophobic air/water interface, which leads to fibrillar aggregation. This is done on a rotating mixer with insulin vials connected to its face in a circumferential orientation (other mixers and methods have been used as well). An example of a rotating mixer is shown in FIG. 1. Light scattering, transmittance, or ThT fluorescence is measured at least 1 time every day, and a 3-fold increase in turbidity or fluorescence over baseline indicates the start of the exponential fibril growth.

The third test measuring chemical stability is assayed by High-Performance Liquid Chromatography (HPLC) and Size-Exclusion Chromatography (SEC) on samples incubated in sealed vials at temperatures ranging from 4° C. to 50° C. Samples are taken for testing at 2 weeks, 4 weeks, 8 weeks, and 12 weeks. Total insulin concentration, Desamido A21, and Related Substances are measured by HPLC following the respective USP monograph (e.g. Insulin Injection or Insulin Lispro injection monographs) or variations of that method. The percentage of HMWP can be estimated from the previous HPLC method, or it is more accurately measured by following USP monograph 121.1, an SEC method, or a variation of it.

### Example No. 2—Optimization of Surfactant Concentration

Poloxamer 188 is an FDA-approved (originally approved as a therapeutic agent to reduce blood viscosity before transfusions and now used as a biocompatible ingredient in many pharmaceutical and cosmetic products) surfactant of increasing use in formulations of antibody therapeutics in the last two decades because it provides strong protection against the physical aggregation of molecules, and it is more chemically stable than Polysorbates (traditional surfactants). The typical concentration range for Poloxamer 188 in antibody therapeutics spans from 0.1 to 1 mg/mL. To study the effect of an increased surfactant concentration on the physical stability of a U500 Human Insulin formulation, a standard U500 formulation (Std.) containing 1 mg/mL of Poloxamer 188 was chosen as a comparator. The complete formulation consisted of 20 mg/mL glycerol, 1 mg/mL Poloxamer 188, 2.9 mg/mL phenol, and 0.085 mg/mL zinc. The insulin API was pre-treated in 50 mM NaOH for 30 minutes before neutralization and the subsequent addition of excipients. The study consisted of 5 formulations where the only variable was Poloxamer 188 concentrations ranging from 1 mg/ml (the baseline “Std.”) to 2.5 mg/mL, as shown in FIG. 2. In FIG. 2, the upper and lower boundaries of boxes respectively indicate first and third quartiles (25th and 75th percentiles), upper and lower whiskers demarcate max and min values, and Horizontal lines correspond to median. The vertical positions of the dots represent individual values from the study sample. P values are shown that describe the probabilities that difference between the formulations that are being compared (end points of “boxes” above pairs of formulations) is the result of random error.

Since there is no insulin or insulin analog formulation containing Poloxamer 188, we used an additional comparator based on Insuman Implantable U400 (Sanofi), a high-concentration insulin comprising 0.01 mg/ml of Poloxamer 171. Insuman Implantable U400 is no longer commercially available, nor is its Poloxamer 171 excipient. We designed a similar formulation as a comparator and called it Unsuman. This formulation contains 0.01 mg/ml of Poloxamer 181 (closely related to Poloxamer 171), 20 mg/ml of glycerol, 50 mM Tris, 2.7 mg/ml of phenol, and 0.1 mg/ml of Zinc. The physical stability was evaluated by measuring the lag time in a Nephelometer under 300 RPM agitation and at 37° C. A 96-well plate was used, containing one Teflon bead in each well and 250 μL of the formulation. Each formulation was distributed across 14 wells (independent replicates). As seen in FIG. 2, the lag time to fibrillation of the Std. was 2.5 times higher than Unsuman, and the highest increase in stability was seen by between 1.5 mg/mL and 1.75 mg/ml of Poloxamer 188 >2 times the lag time of Std. The impact Poloxamer 188 was additive to the impact of the pre-treatment with 50 mM NaOH (the high pH treatment described above).

### Example No. 3—Optimization of Preservative Concentration

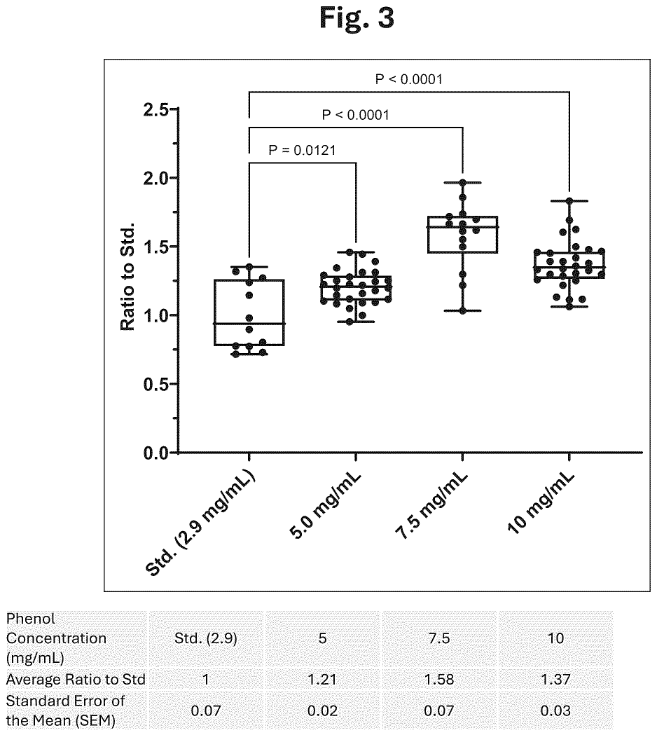

To study the effect of increased phenolic preservative on the physical stability of a U500 Human Insulin formulation, a standard U500 formulation (Std.) was chosen as a comparator. This “Std.” formulation contained 20 mg/mL glycerol, 1 mg/mL Poloxamer 188, 2.9 mg/mL phenol, and 0.085 mg/mL zinc. The insulin API was pre-treated in 50 mM NaOH for 30 minutes before neutralization and subsequent addition of excipients. The physical stability was evaluated by measuring the lag time in a Nephelometer under 300 RPM agitation and at 37° C. A 96-well plate was used, containing one Teflon bead in each well and 250 μL of the formulation. Each formulation was distributed across 14 wells (independent replicates). The Std. was compared to 3 formulations containing the same excipient mix as Std. except for increasingly high concentrations of Phenol: 5.0 mg/dl, 7.5 mg/dL and 1.0 mg/dL. As shown in FIG. 3, all 3 formulations significantly increased the physical stability of the formulation, with 5.0 mg/dl increasing the stability by 21% (Average Ratio to Std. of 1.21), 7.5 mg/dl by 58% and 10 mg/dL by 37%. This effect was additive to the effects of pre-treatment with 50 mM NaOH, and of including Poloxamer 188 in the formulation as described in Example 2. As was the case in FIG. 2, in FIG. 3 upper and lower boundaries of boxes respectively indicate first and third quartiles (25th and 75th percentiles), upper and lower whiskers demarcate max and min values, and horizontal lines correspond to median. The vertical positions of the dots represent individual values from the study sample. P values are shown that describe the probabilities that difference between the formulations that are being compared (end points of “boxes” above pairs of formulations) is the result of random error.

### Example No. 4—Optimization of Osmolyte Concentration

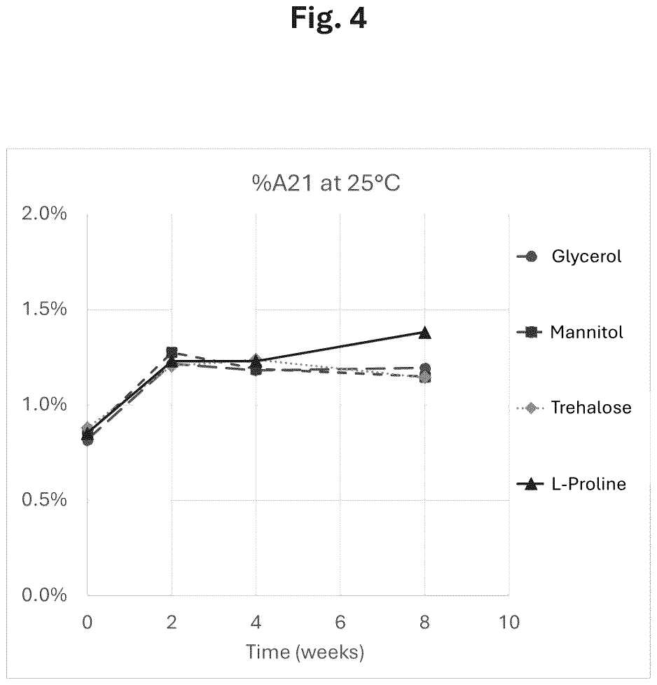

FIGS. 4-9 relate to studies on osmolyte concentration. These are next described. A variety of osmolytes were evaluated. Sucrose caused increased chemical degradation of insulin at high temperatures (11.64% of related substances at 2 weeks and 37° C.), and Betaine caused a slight decrease in the physical stability of insulin.

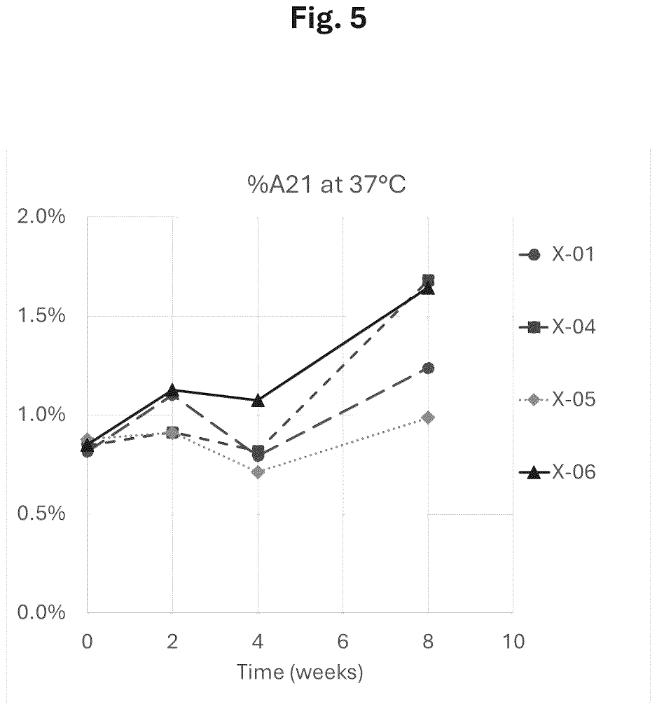

To study the protective effect of osmolytes in comparison with glycerol, a shelf stability study was designed at three temperatures: 5° C., 25° C. (room temperature) and 37° C. (body temperature). The standard formulation used as a comparator consisted of 20 mg/mL glycerol, 1 mg/mL Poloxamer 188, 2.9 mg/mL phenol, and 0.085 mg/mL zinc, with the API (at U500 concentration) pre-treated in 50 mM NaOH for 30 minutes before neutralization and subsequent addition of excipients. The 20 mg/ml of glycerol in the standard formulation corresponds to approximately 217 mM. Since a higher concentration is desirable for a protective osmolyte yet isotonicity was still desired, a 250 mM concentration was chosen for the osmolytes used to replace glycerol in the three alternative formulations: Mannitol, Trehalose, and L-Proline. The chemical degradation of insulin was assessed using reversed-phase high-performance liquid chromatography (RP-HPLC) to quantify its known degradation products including Desamido-A21 (A21), other related substances (ORS), and high molecular weight proteins (HMWP). An HPLC method capable of measuring all three degradation products was used. The formulations were incubated in multiple 2 ml vials, and one vial of each was pulled at every time point studied, which were 0, 2, 4, 6, and 8 weeks. The resulting data presented in FIGS. 4 through 9 show that all three alternative osmolytes considerably slow down chemical degradation compared to glycerol. Regarding A21, FIG. 4 shows osmolyte impact at 25° C., and FIG. 5 shows it at 37° C. These figures illustrate that all four formulations showed similar trends, with the most significant difference observed at 8 weeks at 37° C. (FIG. 5), where Glycerol showed 1.24%, Mannitol 1.68%, L-Proline 1.64%, and Trehalose showed the best result with only 0.99% of A21. We note, however, that A21 is essentially equipotent with insulin and often counted as part of total insulin content for that reason.

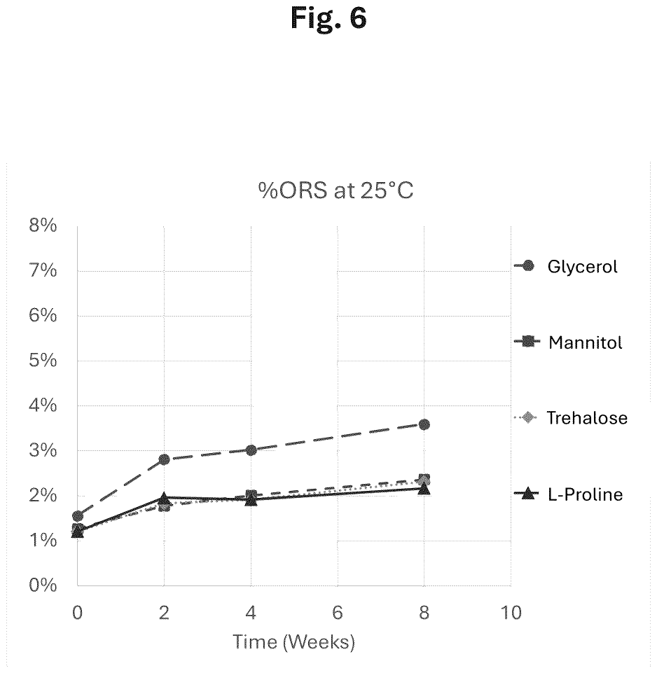

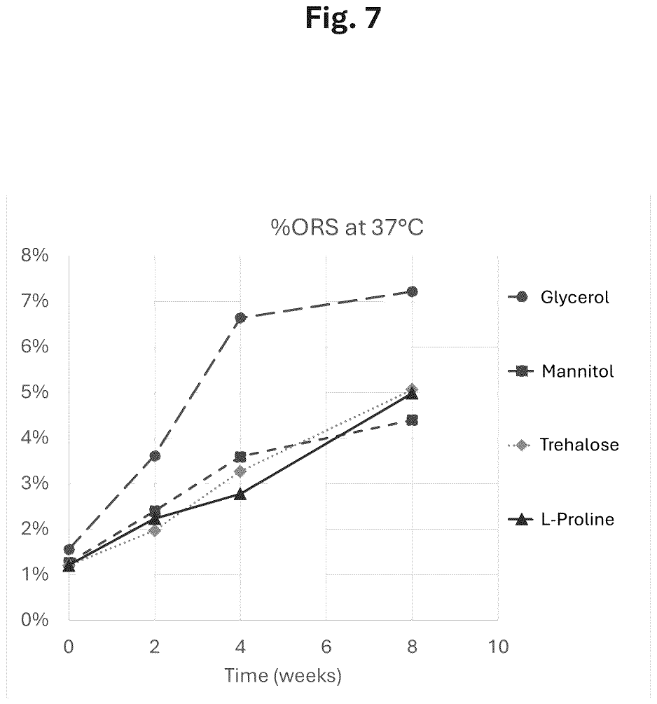

FIGS. 6 and 7 show the results of osmolyte impact studies on “other related substances” (“ORS”). As shown, the formulations containing the alternative osmolytes behave similarly with at least 2% less ORS than the glycerol-containing formulation after 8 weeks at 37° C.

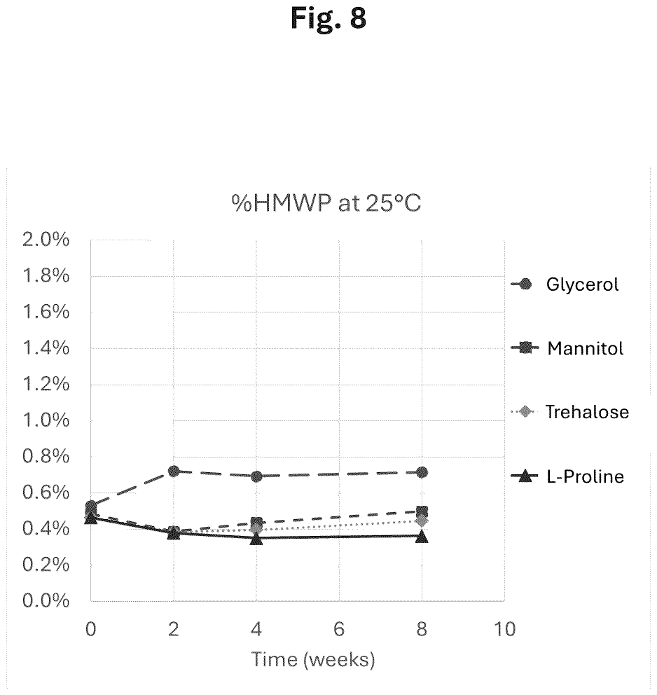

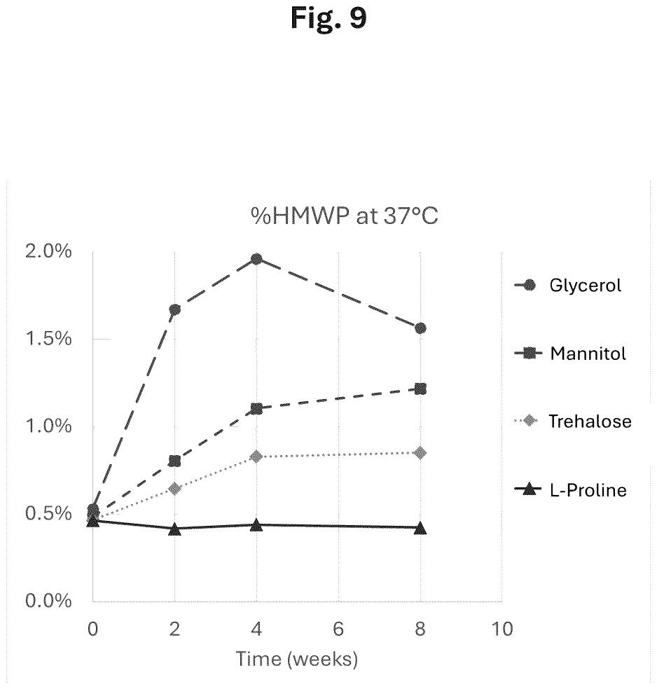

The biggest individual difference is seen with osmolyte impact on HMWP, shown in FIGS. 8 and 9, where L-Proline is considerably superior, accumulating only 0.42% after 8 weeks at 37° C. (no change from the baseline of 0.46%), as shown in FIG. 9, compared with 0.85% by Trehalose, 1.22% by Mannitol, and 1.56% by Glycerol, as also therein shown.

In summary, Proline showed the biggest improvement in protection against chemical degradation. Surprisingly, proline completely prevented problematic degradation into HMWP, with no increase in HMWP after 4 weeks at both 25 and 37° C. Such a dramatic improvement in HMWP has never been reported before in insulin formulation studies to the knowledge of the inventors.

### Example No. 5—Optimization of High pH Pre-Treatment

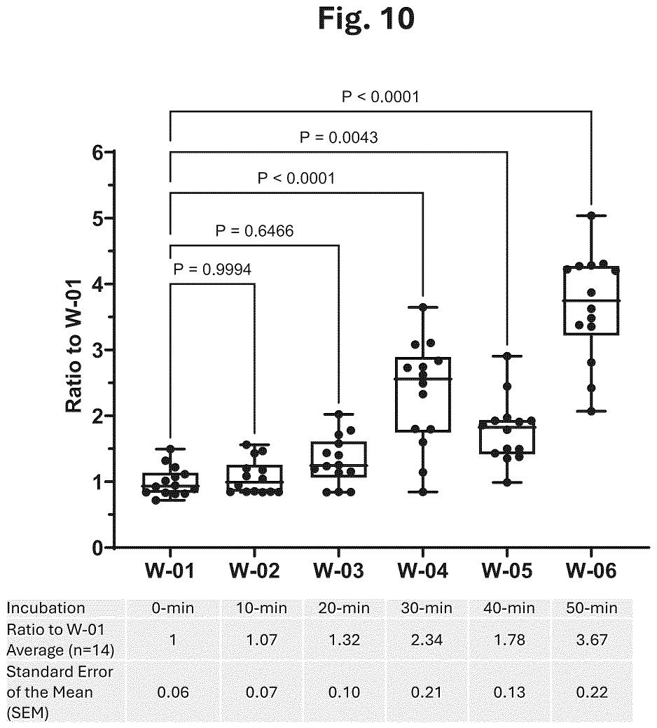

FIGS. 10-17 illustrate optimization of the high pH pre-treatment of insulin API, and these are next described. High-pH pre-treatment of insulin API, when applied for over 20 minutes showed a linear increment of physical stability in both assays. However, after 50 minutes of high-pH pre-treatment, the time zero levels of related substances significantly increased. Because high pH pre-treatment did not show any changes in initial chemical degradation below 40 minutes, a 20-40 min range was found to be optimal.

A study was performed to optimize the high-pH treatment of the insulin API. Six U-500 formulations, identical in composition but differing in pH treatment duration (from 0 minutes to 50 minutes, as shown in FIG. 10) Each formulation contained 20 mg/mL glycerol, 1 mg/mL Poloxamer 188, 2.9 mg/mL phenol, and 0.085 mg/mL zinc. The insulin API was solubilized in 50 mM NaOH and mixed gently for the respective time before neutralizing to a pH of 7.4. The excipients were then added after the solution was neutralized.

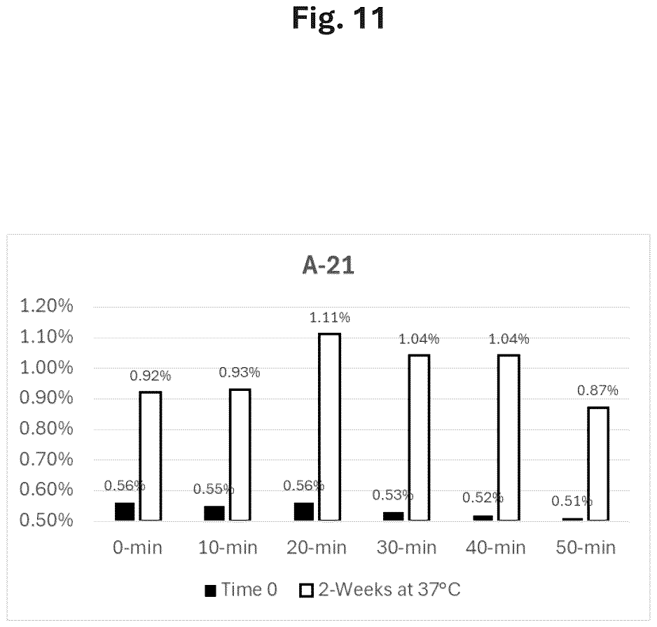

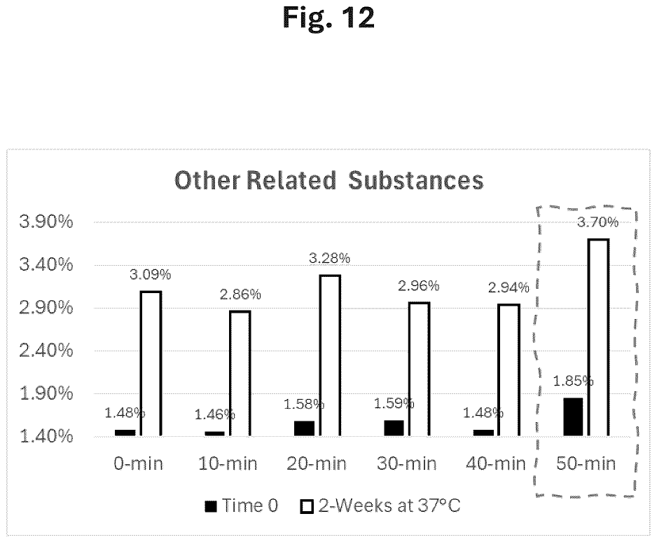

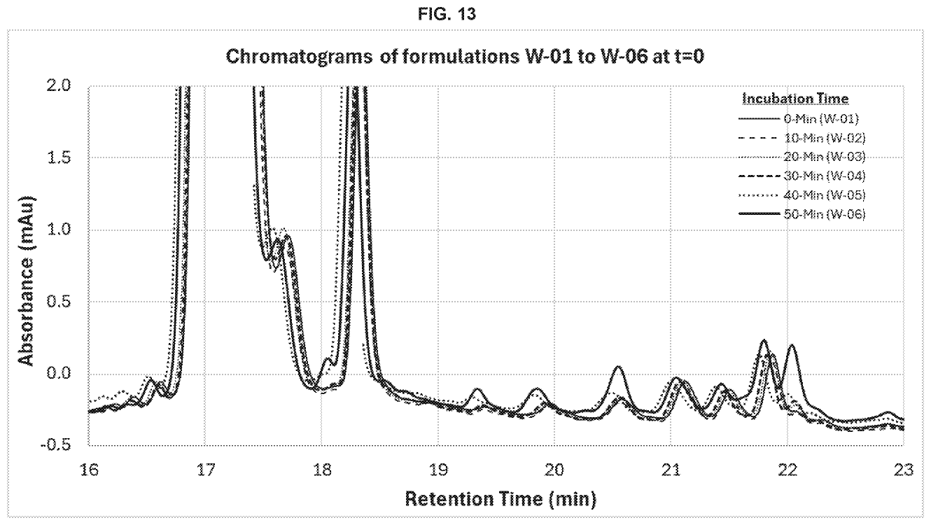

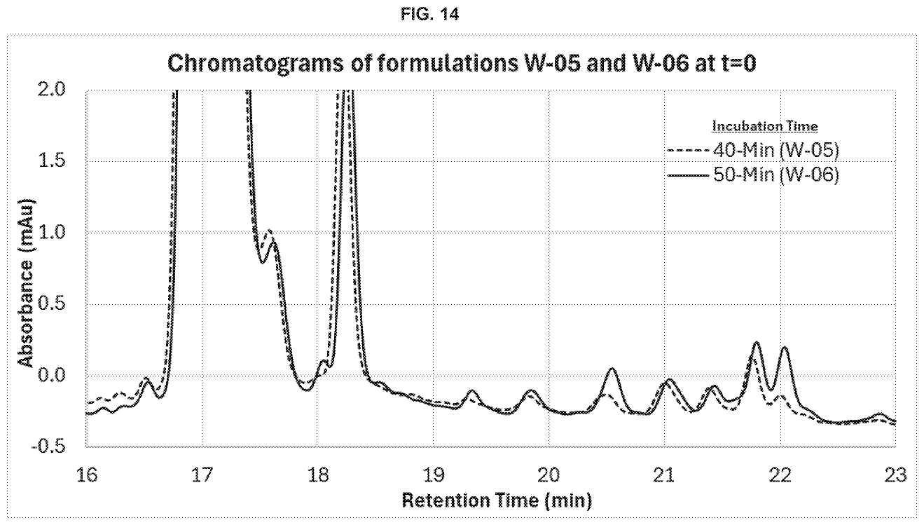

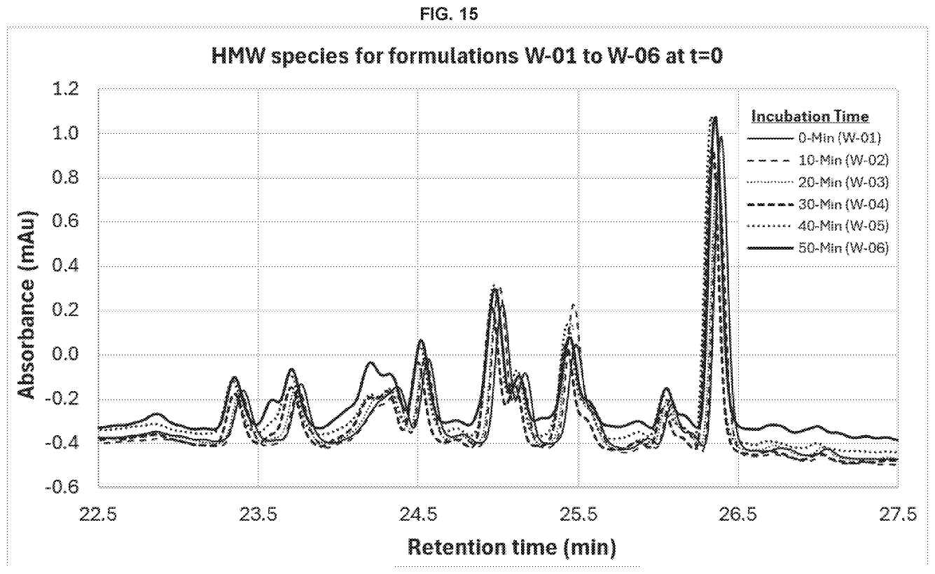

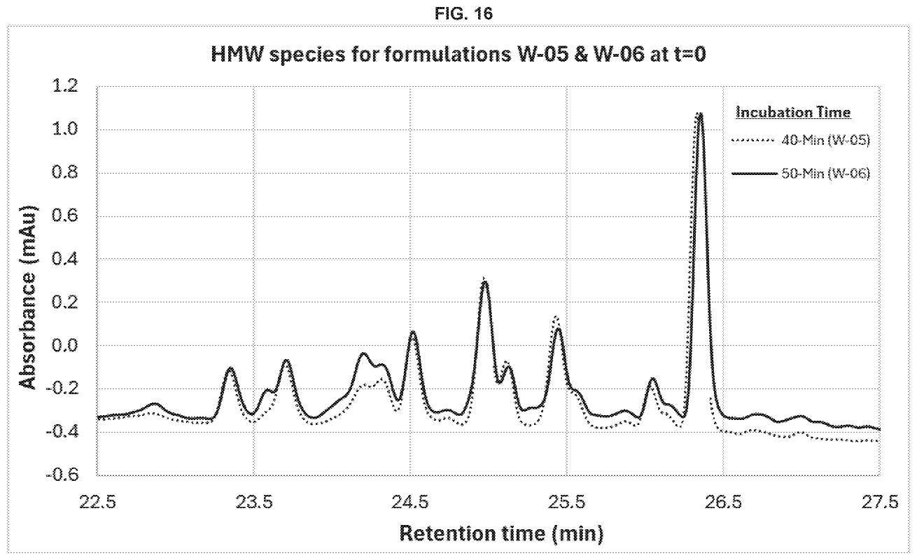

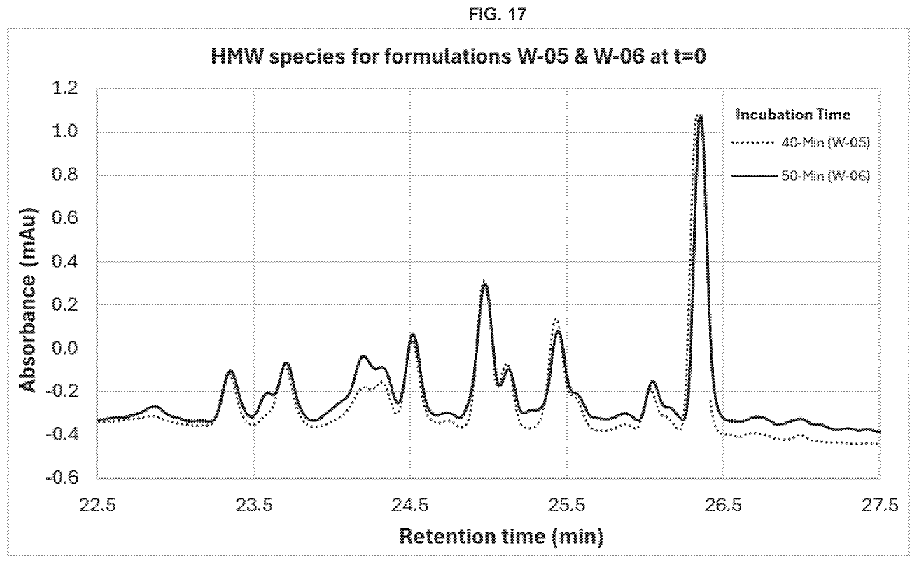

The physical stability was evaluated by measuring the lag time (the time it takes to initiate amyloid-like fibrillation) in a Nephelometer (plate reader equipped with a turbidity measurement capability) under 300 RPM agitation and at 37° C. A 96-well plate was used, containing one Teflon bead in each well and 250 μL of the formulation. Each formulation was distributed across 14 wells, which were considered independent replicates, as the fibril formation process is a stochastic event with high well-to-well variability. The results are shown in FIG. 10. As shown, all treatments for over 20 minutes showed an increase in stability, as measured by average lag time, with the 50-minute treatment displaying the most significant enhancement, increasing the average lag time by 3.67 times compared to the control. In FIG. 10, the upper and lower boundaries of boxes respectively indicate first and third quartiles (25^{th} and 75^{th} percentiles), upper and lower whiskers demarcate max and min values, and Horizontal lines correspond to median. The vertical positions of the dots represent individual values from the study sample. P values are shown that describe the probabilities that difference between the formulations that are being compared (end points of “boxes” above pairs of formulations) is the result of random error. However, the 50-minute treatment showed an increase in chemical degradation not seen in any other treatment duration up to 40 minutes. As shown in FIGS. 11, 12 and 13 respectively, chemical degradation was studied by high-performance liquid chromatography (“HPLC”) measurements of Desamido A21 insulin (A21), other related substances (“ORS”), and HMWP. After the formulations were prepared an aliquot was taken for HPLC measurements, and an additional aliquot was placed in a 2 mL vial and incubated at 37° C. for 2 weeks before again measuring by HPLC chemical degradation. The two-week time point was to determine if the treatment had any chemical effect on insulin that was not evident at the time of preparation. There was no trend in A21 or HMWP across all six formulations (FIGS. 11 and 13), and no significant difference when compared to the control (0-minute treatment). There was a small but significant increase in ORS at both time 0 and 2-week for the formulation that received 50 minutes of high-pH treatment, as shown in FIG. 12. There was a 1.85% of RS versus 1.52% of average in the other 5 formulations at time 0, with 3.70% vs 3.03% after 2 weeks at 37° C. A comparison of the chromatographic profiles confirmed a noticeable increase in RS for the 50-minute treatment compared to the other formulations [W-06, shown in FIGS. 14 and 15] and also a small increase in HWMP [W-06, shown in FIGS. 16 and 17]. Based on this data, it was concluded that 40 minutes was an appropriate high-pH treatment time that does not increase chemical degradation.

### Example No. 6—Insulin Formulation for Intraperitoneal (IP) Delivery

Insulin delivered directly into the peritoneal cavity (the peritoneum is a membrane that encloses several organs in the abdomen) overcomes many of the limitations of subcutaneously (SC) delivered insulin. Veins in this cavity drain into the hepatic portal vein, so insulin delivered into this cavity follows the same route as insulin released by the pancreas. This contrasts with SC-delivered insulin where most of the dose remains in the periphery—leading to higher than typical peripheral insulin levels and action in fat and muscle (a condition called “Peripheral Hyperinsulinemia”), heightened risk of hypoglycemia, and weight gain (insulin is a growth hormone that expands adipose tissue). There is evidence to suggest that IP delivery significantly reduces the risk of hypoglycemia. In addition, the extensive vasculature in the peritoneal cavity enables much faster absorption of insulin (e.g., 2.3× in one study) than is possible for SC delivered insulin.

Accessing the peritoneal cavity requires an implanted catheter and, ideally, an implanted pump with a reservoir of insulin inside the body. An insulin formulation stored inside the body must be significantly more stable than the insulin formulations currently available. It should also be highly concentrated so that a small reservoir can hold a large number of units so transcutaneous refills can be less often. In embodiments, an example insulin formulation optimized for such a use may be used, although such a formulation could also be used for SC delivery, for example, to treat highly insulin-resistant patients. In this example, regular human insulin was used as the API at a U500 (500 International Units per mL or 17.4 mg/mL) formulation with prolonged stability at room or body temperature. The insulin was solubilized in 50 mM sodium hydroxide (NaOH) with subsequent gentle mixing for 30 (20-40) minutes. The insulin solution was then adjusted to pH 7.0-7.8 utilizing sulfuric acid (H₂SO₄). Sulfuric acid was used to mitigate potential corrosion of implanted pump surfaces by CI-ions from HCl.

The insulin was next formulated to contain 0.55% (0.35 to 0.75%) phenol as a preservative, 1.65 (1.5 to 1.8) mg/ml of Poloxamer 188 as a surfactant, 30 (25 to 35) mg/ml of L-Proline as an osmotic agent, and 0.085 (0.08 to 0.10) mg/ml of Zn as ZnSO4, rather than ZnCl, to again avoid CI-ions. To improve the solubility of the API, the formulation contains no pH buffer other than the high concentration of the API itself.

This formulation was named PI-U500, and three independently made lab-scale batches were prepared to study its physical and chemical stability, as detailed below. A U500 comparator was created based on Insuman Implantable U400 (Sanofi). Insuman Implantable U400 is the only insulin drug that has been approved (EU only) for intraperitoneal administration by an implantable pump, however it is no longer commercially available. Its Poloxamer 171 excipient is also no longer commercially available. We designed a similar formulation as a comparator and called it “Unsuman.” Unsuman has a higher concentration of insulin (U500), and like Insuman, it contains 0.01 mg/ml of a surfactant—in this case Poloxamer 181—which is closely related to Poloxamer 171. Like Insuman, it also contains 20 mg/ml of glycerol, 50 mM Tris, 2.7 mg/ml of phenol, and 0.1 mg/ml of Zinc. We used it as a benchmark for stability, as Insuman was sufficiently stable to be approved for in-use storage in an implantable pump (i.e., at body temperature) for up to 45 days.

### Physical Stability

In general, the onset and rate of fibrillation increase with elevated exposure to temperature, agitation, or hydrophobic surfaces (such as an air-water interface). As is common practice with insulin stability studies, to shorten the duration of experiments we elevated exposure to these fibril inducers by two (2) different methods:
1. Traditional physical stress test. The formulation was exposed to continuous agitation in a nephelometer plate reader at 37° C. Fourteen (14) 150 μl samples of each of the four insulin formulation batches were dispensed into a 96-well plate. A Teflon bead was added to each well to increase mechanical stress as the beads rapidly hit the walls of the plastic plate. The top of the plate was sealed with plastic film. The plate was placed on a Thermo Nepheloskan capable of measuring scattered light (nephelometry) then exposed to constant agitation at 300 RPM while being kept at 37° C. Nephelometer measured turbidity signal was every 5-15 minutes; a 3-fold increase in turbidity over baseline indicated the start of the exponential fibril growth; the time to this start is called the “lag-time.” Lag time was then aggregated across the 13 samples of each formulation.
2. Air-water exposure test. Because the primary driver of fibrillation in implanted pumps is expected to be exposure to bubbles in the reservoir rather than aggressive agitation of the pump, the second test was designed to maximize air/water interface exposure. In this test six (6) crimp-sealed 2 mL HPLC vials per batch, each containing 500 μL of insulin formulation, were agitated in a rotatory fashion at 80 RPM using a custom-built rotary mixer (shown in FIG. 1) placed in a 37° C. incubator. As the formulation repeatedly poured past the 1.5 ml of air in the vial, the system stimulated dynamic interaction of the insulin formulation with a hydrophobic air/water interface. Turbidity through light scattering was measured at least 1 time every day, and a 3-fold increase in turbidity over baseline indicated the start of the exponential fibril growth.

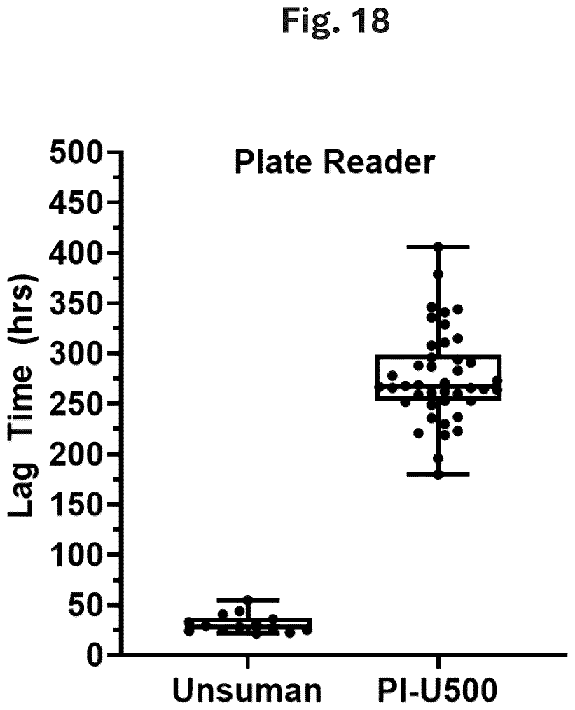

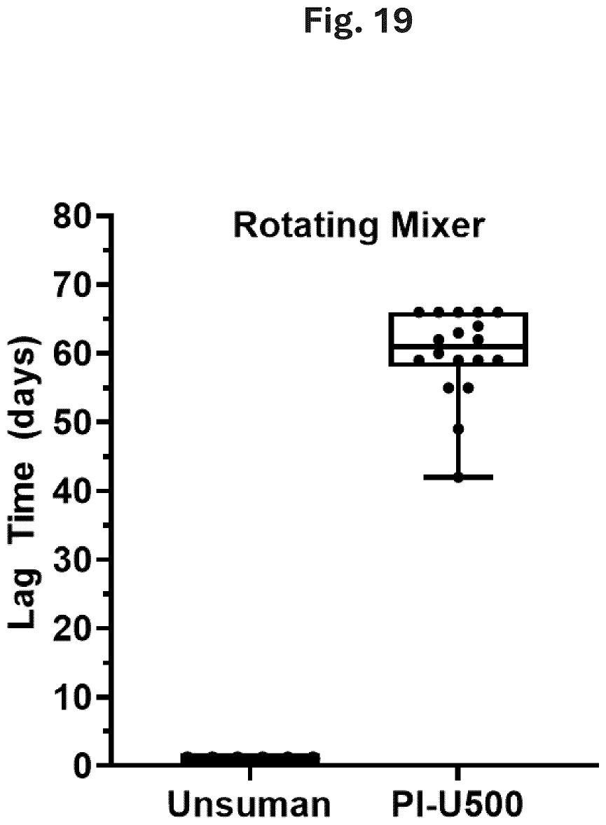

PI-U500 was significantly and consistently more physically stable than Unsuman. In the plate reader, PI-U500 had a lag time of 276.8±7.1 hours, meaning it took over 11 days of constant vigorous agitation at 37° C. for it to start fibrillating. In comparison, Unsuman's lag time was almost 9 times lower (31.52±2.5 hours), beginning to fibrillate on the second day of agitation. This is shown in FIG. 18. Notably, in the rotating mixer, the difference was even bigger, amounting to 60 times the improved resistance to fibrillation for PI-U500 over Unsuman, as shown in FIG. 19. This may be because the high hydrophilicity surfactant present in a high concentration in PI-U500 is more effective at coating hydrophobic surfaces (like the air/water interface) than are low hydrophilicity surfactants at low concentrations (like those in Unsuman and Insuman Implantable U400) and therefore protect insulin from the fibrillogenic effect of contacting those surfaces. The rotating mixer is designed to expose the formulations to an air/water interface, so the surfactant's protective effect is more evident than it is in the plate reader test that has a teflon bead intensely hitting the walls of the wells, and where the main driver of fibrillation is physical smashing (against which surfactant quality offers less protection) instead of hydrophobic surface exposure. It may also be that hydrophobic surface exposure must be sustained for some critical period of time (e.g., less than 1 second but more than a microsecond) for the exposure to cause insulin to unfold and present its hydrophobic core (thus becoming susceptible to aggregation) and that the plate reader's rapid motion means that the exposure periods are too brief.

It is noted, once again, in each of FIGS. 18 and 19, that the upper and lower boundaries of boxes respectively indicate first and third quartiles (25th and 75th percentiles), the upper and lower whiskers demarcate max and min values, and Horizontal lines correspond to median. The vertical positions of the dots represent individual values from the study sample. P values are shown that describe the probabilities that difference between the formulations that are being compared (end points of “boxes” above pairs of formulations) is the result of random error.

### Chemical Stability

Insulin formulations undergo chemical degradation that increases with time at a temperature-dependent rate. The most common degradation products are the “related substances” generated by deamidation of asparagine (ASN) to aspartic acid (ASP) at A21, A18, or B3, or the oxidation of histidine (HIS) at B5 or B10. Typically, Desamido A21 is reported separately from the other related substances, which are aggregated as other related substances (“ORS”), because A21 is considered innocuous. The other main degradation products are covalent aggregates (e.g., dimers and trimers). Called high molecular weight proteins (HMWP), these are different from fibrils because they grow linearly with time rather than exponentially. These species generally form through transamidation and disulfide exchange, which is not catalyzed by acidic incubation conditions but is accelerated by elevated temperatures under conditions where dimers or higher-order insulin oligomers are present, including in insulin powder. HMWPs are considered more problematic than related substances because (a) they pose a greater risk of immunogenicity and (b) unlike related substances, their presence has a big impact on potency because HWMP have significantly lower receptor binding affinity.

To evaluate the chemical stability of each formulation, one (1) HPLC crimp sealed vial per time point, per temperature, was filled with 1 mL of formulation and then incubated at 5° C., 30° C., 37° C., and 50° C. for 4, 8, and 12 weeks.

To detect chemical degradation, an RP-HPLC method was developed to measure total insulin concentration and insulin's main degradation products (A21, RS, and HMWP) in a single run. This method is based on the USP monographs “Insulin human Injection” and “Insulin lispro injection”, but modifications to the monographs allow for the calculation of insulin concentration and HMWP without the need for a separate size-exclusion chromatography (SEC) method for quantifying HWMP.

Single replicates of each of the three PDI-made batches of PI-U500 and of the single PDI-made batch of Unsuman were incubated and then evaluated for their chemical stability.

### High Molecular Weight Proteins

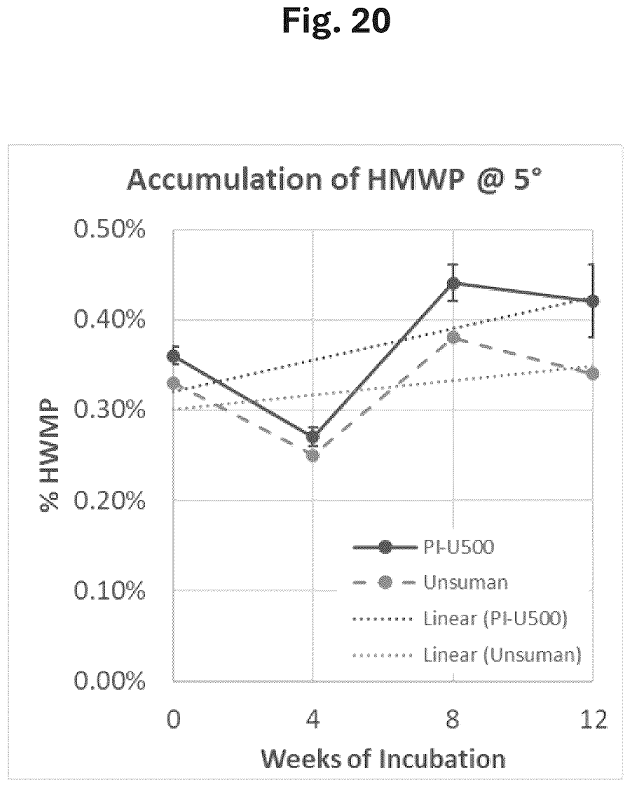

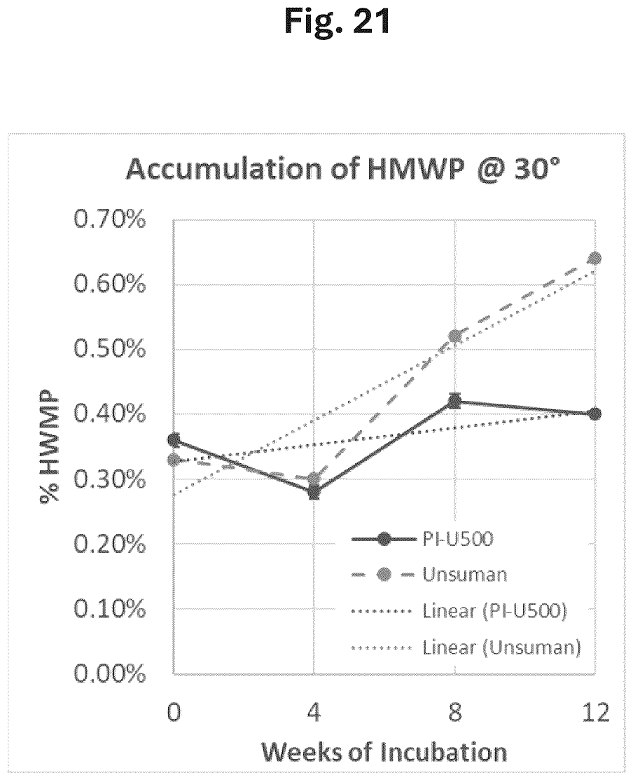

HWMP are the most important species to minimize because they are impotent and immunogenic. The rate of growth of these species increases with temperature, creating a challenge for any insulin formulation utilized in an implanted AID. These studies demonstrated that HWMP formation for PI-U500 was roughly the same as that for Unsuman at refrigerated storage conditions (5° C.) but was significantly slower at higher temperatures, as shown in FIGS. 20-23, which all show results of PI-U500 HMWP accumulation at various temperatures. PI-U500's lower rate of HMWP formation becomes increasingly pronounced as incubation temperatures increase, with the most dramatic difference occurring above 37° C., as shown in FIGS. 22-23.

### Other Related Substances and Desamido A21

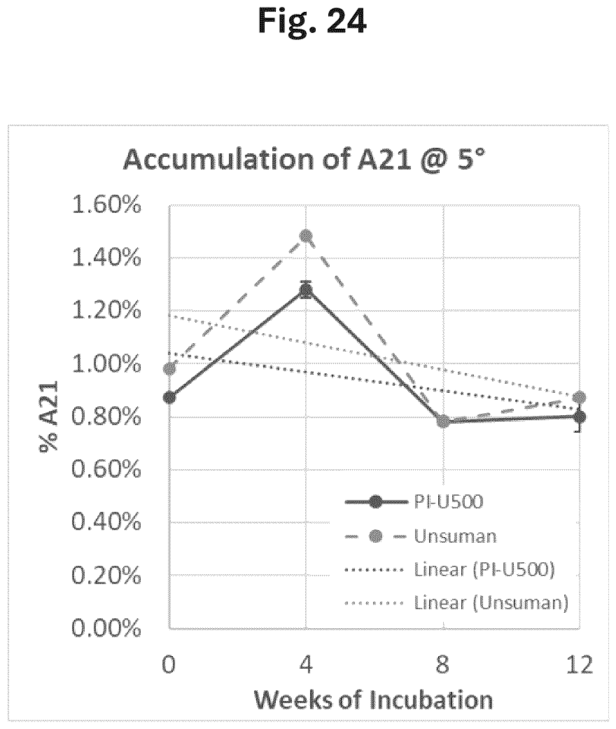

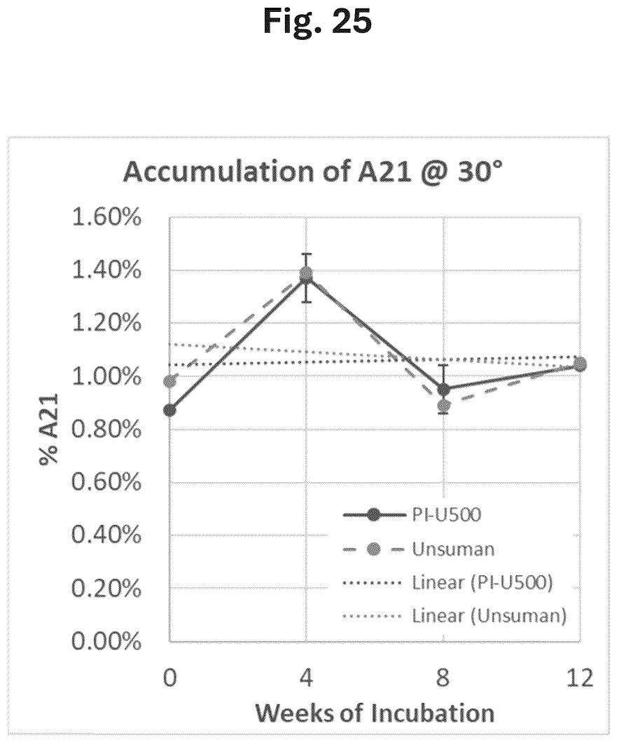

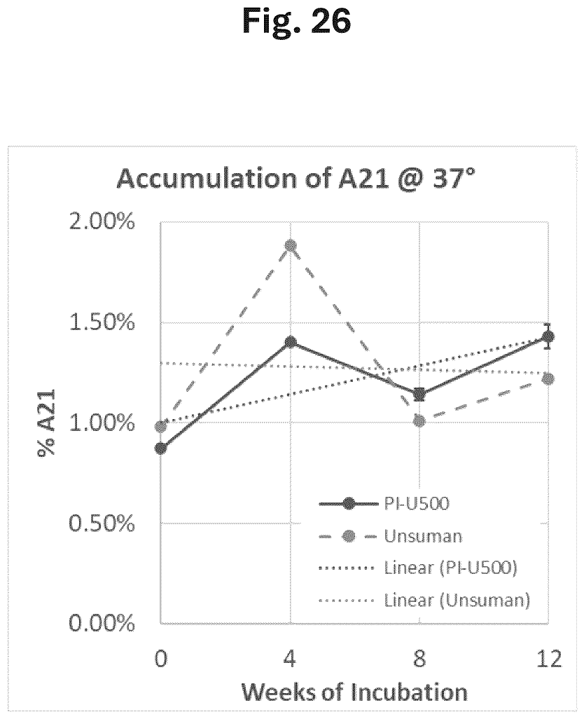

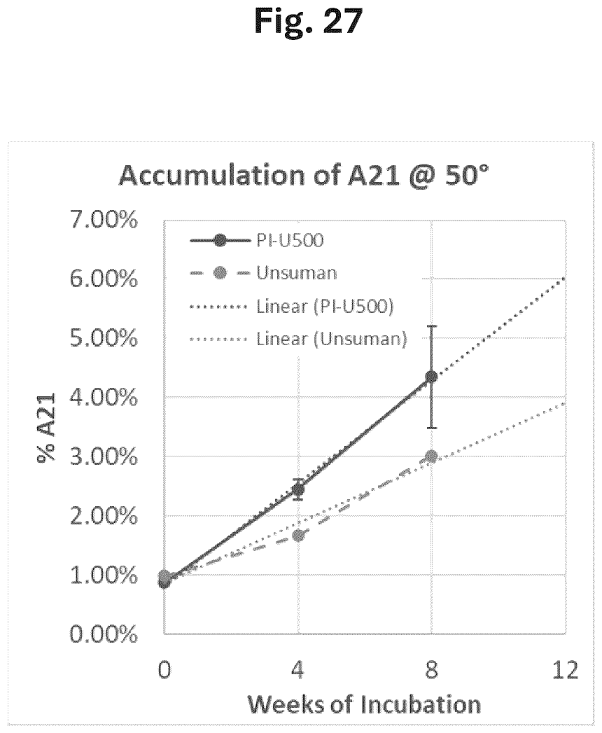

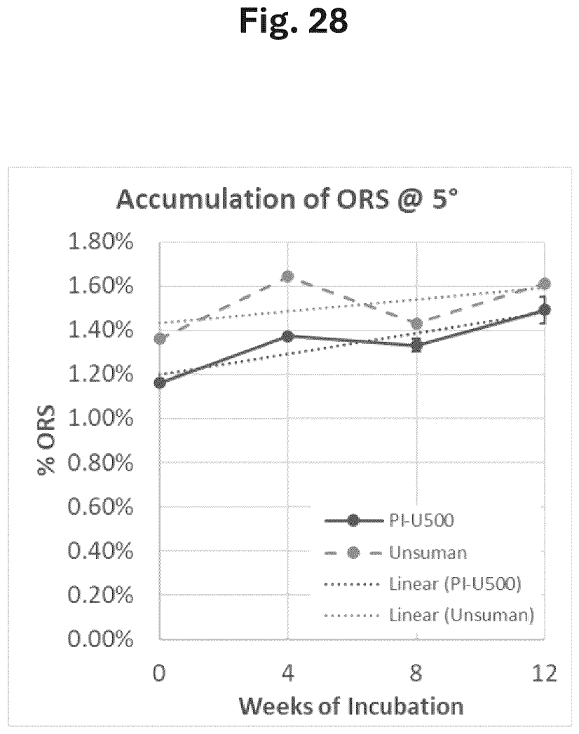

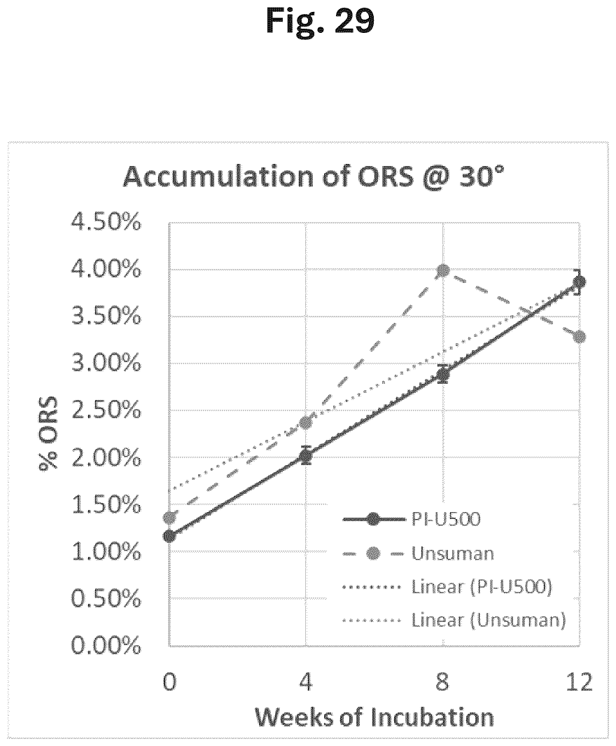

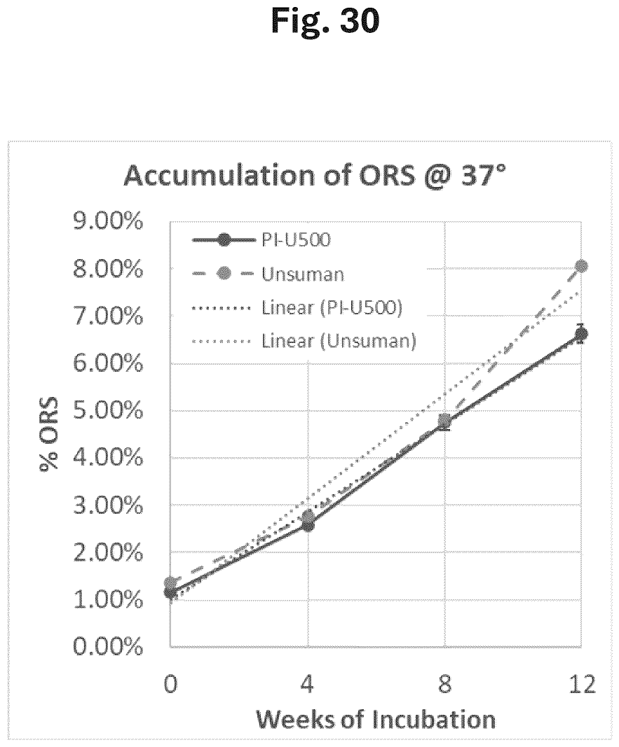

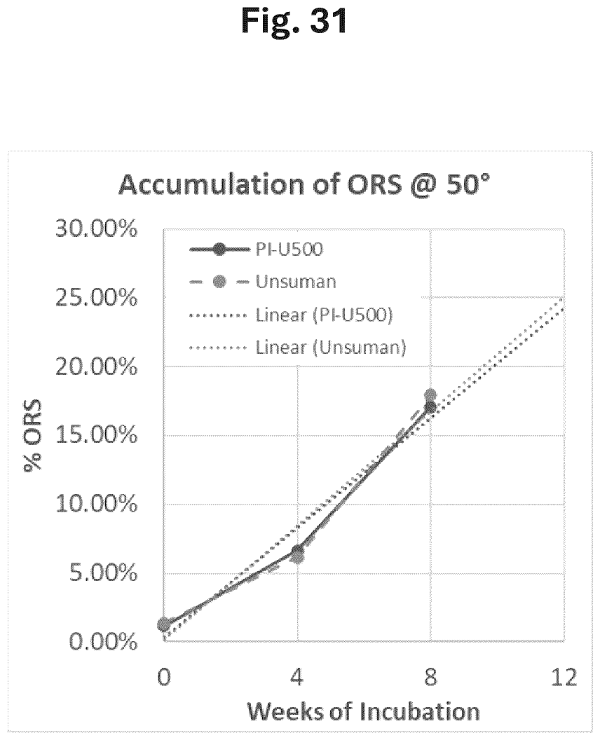

FIGS. 24-31 show PI-U500 Desamido A21 accumulation at four temperature levels, and then PI-U500 ORS accumulation at the same temperature levels. As shown in these plots, PI-U500 shows a similar resistance to ORS degradation as Unsuman, with a possible reduction in ORS seen at 37° C. after 12 weeks. For Desamido A21, PI-U500 is also not better than Unsuman and may even have a higher rate of A21 formation at elevated temperatures. Although not a notorious improvement over Unsuman, A21 and ORS are not chemical degradation products of concern, as A21 is routinely included in the Potency (Assay) calculation of insulin as part of the main insulin content and some ORS may also be included in that calculation if they are specifically identified and their relative potency calculated.

### Example No. 7—Safety and Efficacy of Formulation

In this example, the example PI-U500 formulation was tested in vivo in both rats and dogs; together the studies show that the enhanced formulation does not adversely affect either the safety or efficacy of the insulin.

In rats, after 14 days of daily injections of both PI-U500 and vehicle, no adverse local intolerances were seen. The study consisted of one saline control group (Group 1), two Vehicle groups (Groups 2 and 3), one Humulin R-treated (Group 4), and two Test Article-treated groups (Groups 5 and 6). Each Main group consisted of 10 rats/sex. Groups 1, 4, 5, and 6 also had animals designated for glucodynamic (GD) evaluations to confirm effectiveness of delivery. All study animals were dosed once daily via IP administration for 14 days. The study design is summarized in Table D:

TABLE D
Rat Study Design

| | | | | | Dose | Dose |
|---|---|---|---|---|---|---|
| | | Number of | | Dose | Con- | Volume |
| Study | | Rats/Sex | | Level | centration | (mL/ |
| Group | Treatment | Main | GD | (U/kg)ª | (mg/mL) | kg) |
| 1 | Saline Control | 10 | 6 | 0 | 0 | 0.2^{b} |
| 2 | Vehicle-Low Vol. | 10 | 0 | 0 | 0 | 0.2^{b} |
| 3 | Vehicle-High Vol. | 10 | 0 | 0 | 0 | 2.0^{c} |
| 4 | Humulin R | 10 | 6 | 100 | 17.4 | 0.2 |
| 5 | Portal Insulin- | 10 | 6 | 30 | 17.4 | 0.06 |
| | Low Dose | | | | | |
| 6 | Portal Insulin- | 10 | 6 | 100 | 17.4 | 0.2 |
| | High Dose | | | | | |

Vol. = Volume
^{a}1U = 0.0347 mg Insulin.
^{b}Groups 1 and 2 administered same volume (mL/kg) as Group 6.
^{c}Group 3 administered 10 × volume (mL/kg) as Group 6.

The Test Article [PI-U500] was formulated by the Contract Research Organization (Frontage labs). The Saline Control was 0.9% sodium chloride. The Vehicle was identical to the Test Article except that it contained no insulin (i.e., just the excipients at the same concentration as in the Test Article). The insulin control article was Humulin R U-500 (Eli Lilly and Company, Indianapolis, IN).

The doses administered in this study were significantly higher than those that would be used in humans, who will receive a maximum of 1.2 U/kg/day. A 30 or 100 U/kg dose of PI-U500 (groups 4 & 6) was systemically tolerated by rats but would be fatal in humans. Vehicle group 2 was included to evaluate whether any local intolerance was due to insulin alone. Vehicle group 3 was included to evaluate the local tolerance of the excipients at 10× the quantity of the highest Test Article dose, because such a high dose of the Test Article would have been fatal to the rats.

Results and Conclusions: Once daily IP administration of PI-U500 to male and female Sprague Dawley rats at dose levels of 30 and 100 U/kg/day for 14 consecutive days was well-tolerated, as was daily IP administration of the Vehicle at the concentration equivalents of 100 and 1000 U/day. No Test Article-related mortality or clinical signs occurred during the study. One Humulin R-treated rat died of unknown cause on Day 14 prior to dosing. There were no Test Article- or Vehicle-related effects on body weights, food consumption, clinical pathology, gross pathology, or organ weights. Histology of tissues taken from PI-U500 and Vehicle administered groups were microscopically similar to the saline control except for minimal or mild mononuclear inflammatory infiltrates and hemorrhage, which were similar between the Test Article and Vehicle groups. Based on their nature, these findings were not considered adverse. Changes in blood glucose occurred as expected with the pharmacological activity of PI-U500; blood glucose decreased within 30 minutes of administration of PI-U500 and returned to baseline approximately 6 hours postinjection.

The glucodynamic (GD) and insulin pharmacokinetic (PK) profiles of PI-U500 were compared with the PK/GD profile of “Unsuman” following ultrasound-guided intraperitoneal injection into male beagle dogs. “Unsuman” is a rHI formulation that mirrors the specific excipients and their concentrations in Insuman U400 (Sanofi), the only concentrated insulin formulation marketed (in the EU only) for IP delivery, except that “Unsuman” contains rHI at U-500 concentration. The quantitative composition of the three products is shown in Table E, provided below.

TABLE E
Quantitative composition of test articles in study 24C690Q1

| Ingredient | Insuman U400 | Unsuman U500 | PI-U500 |
|---|---|---|---|
| API | rHI 13.88 mg/ml | rHI 17.35 mg/ml | rHI 17.35 mg/ml |
| Buffer | Tris 6.06 mg/ml | Tris 6.06 mg/ml | None |
| Preservative | Phenol 2.7 mg/ml | Phenol 2.7 mg/ml | Phenol 5.5 mg/ml |
| Tonicity Agent | Glycerol 20 mg/ml | Glycerol 20 mg/ml | L-Proline 30 mg/ml |
| Stabilizing Agent | Zinc 0.068 mg/ml | Zinc 0.085 mg/ml | Zinc 0.085 mg/ml |
| Surfactant | Poloxamer 171 | Poloxamer 181 | Poloxamer 188 |
| | 0.01 mg/ml | 0.01 mg/ml | 1.65 mg/ml |

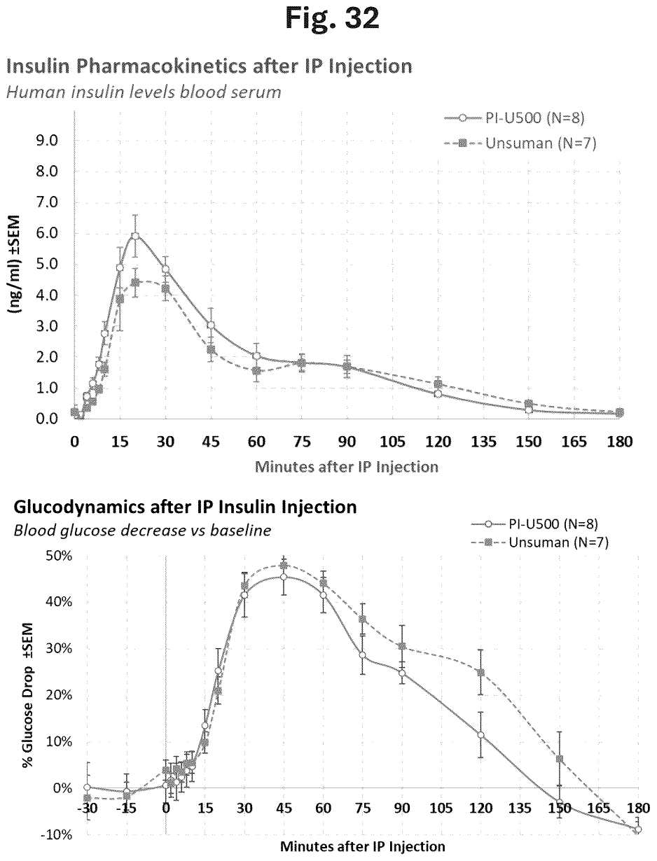

In this study, four non-diabetic male beagle dogs were dosed twice utilizing ultrasound-guided intraperitoneal injection with each of PI-U500 and comparator Unsuman for a total of 8 doses per formulation. FIG. 32 shows plots for the insulin pharmacokinetics and glucodynamics, post-injection.

Human insulin PK was assessed in serum samples from 15 blood samples collected over 3 hours after injection. Human insulin concentrations in each sample were quantitated using a non-validated LC-MS method. PK parameters calculated from the resulting PK curve included maximum measured concentration (Cₘₐₓ), the time of Cmax (Tₘₐₓ), Area Under the PK Curve (AUC₀₋₁₈₀), and time to return to 50% of Cmax (T_{1/2Late}).

GD was assessed by measuring blood glucose (BG) levels 3 times before injection and 15 times over 3 hours after injection, utilizing a glucometer. GD parameters calculated from the resulting GD curve included: maximum blood glucose drop (BG_{drop}), the time of BG_{drop} (Tₘᵢₙ), Area Over the BG curve from 0 minutes to Tₘᵢₙ (AOC_{0-Tmin}), Area Over the Curve 0 to 45 minutes (AOC₀₋₄₅), Area Over the Curve 0 to 150 minutes (AOC₀₋₁₅₀), and time to recovering 50% of the BG drop (T_{BG1/2Late}). Results Summary: 8 studies were completed for PI-U500; only 7 were successfully completed for Unsuman: one study was excluded due to a dose administration error. As shown in FIG. 32, PI-U500 demonstrated a rapid PK/GD profile indistinguishable from that of Unsuman, with an average Tₘₐₓ of 22.5±3.1 min, SE) (Unsuman Tₘₐₓ of 23.6±2.2 min, SE) and T_{1/2late} of <60 minutes. The glucodynamic effects of the insulin formulations is also depicted in FIG. 32 (lower panel) as a percentage drop from baseline blood glucose, where baseline is calculated as the average BG at t=−30, −15, and 0. As shown, PI-U500 shows similar glucodynamic characteristics to Unsuman including a fast onset of action.

### Example No. 8—Insulin Lispro Formulations

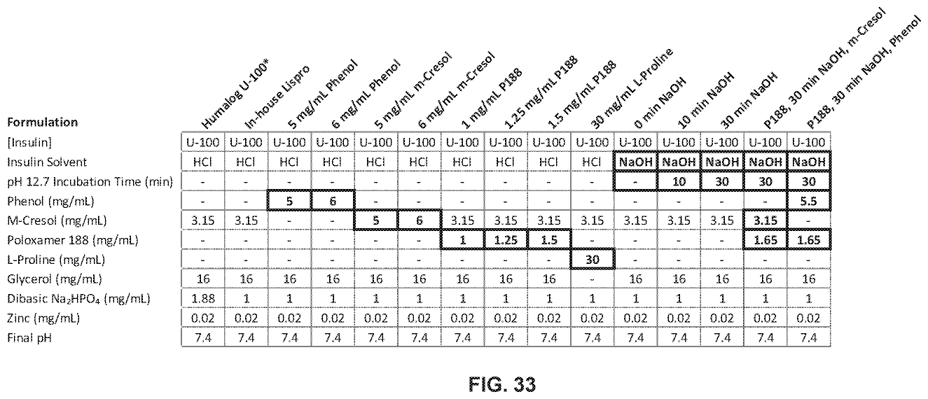

In this example, commercial Humalog U-100 was acquired and in addition fourteen formulations of insulin lispro were made. First, eleven different excipient solutions were made by mixing WFI and specified amounts of excipients, one-by-one in the following sequence: WFI, preservative, surfactant (if applicable), tonicity agents, buffers, zinc. The pH of the excipient solution was adjusted to 7.4+/−0.2. In addition to the standard Humalog-mimicking solution (phenol 3.15 mg/mL, glycerol 16 mg/mL, dibasic sodium phosphate 1 mg/mL, and zinc Q.S. to 0.02 mg/mL), ten excipient solutions were made in which a single substitution (phenol or meta-cresol at 5 or 6 mg/L for the phenol mix, Poloxamer 188 added at 1, 1.25, or 1.5 mg/mL, or L-Proline at 30 mg/mL for the glycerol). In addition, as part of the 11 excipient solutions, one was made with added Poloxamer 188 1.65 mg/mL and one made with both phenol 5.5 mg/ml and Poloxamer 188 1.65 mg/mL. This is illustrated in FIG. 33.

Next, four batches of dissolved lispro were made. One batch was made as follows: appropriate amounts of crystallized insulin lispro to make a U-100 formulation were weighed and mixed with appropriate amounts of acidified WFI; then hydrochloric acid (HCl) was added dropwise while gently mixing for 5-10 minutes until lispro was fully dissolved without further incubation. Immediately following specified incubation times, the lispro solution was adjusted to pH 7.4+/−0.2 with NaOH and dissolved into the first nine of the eleven excipient solutions described previously. Three additional batches were then made as follows: appropriate amounts of crystallized insulin lispro to make a U-100 formulation were weighed and mixed with appropriate amounts of 50 mM sodium hydroxide. The insulin lispro was fully dissolved and incubated with gentle mixing for an additional 0, 10, or 30 minutes, respectively. Formulations were then adjusted to pH 7.4+/−0.2 and dissolved into the standard excipient solution. In addition, the insulin lispro formulation incubated for 30 minutes was dissolved into the last two excipient formulations described above. All 14 formulations were then filtered using 0.2-micron syringe filters, and 0.5 mL aliquoted into 2 ml vials. 0.5 mL of commercial Humalog was also aliquoted into a 2 mL vial for use as a control, also illustrated in FIG. 33.

These vials were tightly capped and incubated on rotating mixer wheels at 20-60 RPM and 37° C. Turbidity readings were taken every 2-48 hours until samples were fully fibrillated, reaching approximately 1000 nephelometer turbidity units (NTU). Fibrillation lag times were defined as 0.2% of the 1000 NTU formazin standard. Parallel samples were dispensed at 200 μL each into a 96-well microplate and run in a nephelometer plate reader set to 37° C., 700-1000 RPM, and orbital shaking. Readings were captured at 10-15-minute intervals for approximately 3 days or until samples entered the plateau phase of fibrillation. Plate reader fibrillation lag times were defined as 1000 basis units above the upper range of the lag phase baseline, with ascending values continuing into log phase. These lag times were visually verified to denote inflection points of sigmoidal curves.

Fibrillation of rotating mixer and plate reader samples was visually verified for cloudiness. In instances where experiments were stopped before some samples began to fibrillate, lag times are defined as the experimental endpoint.

### Results

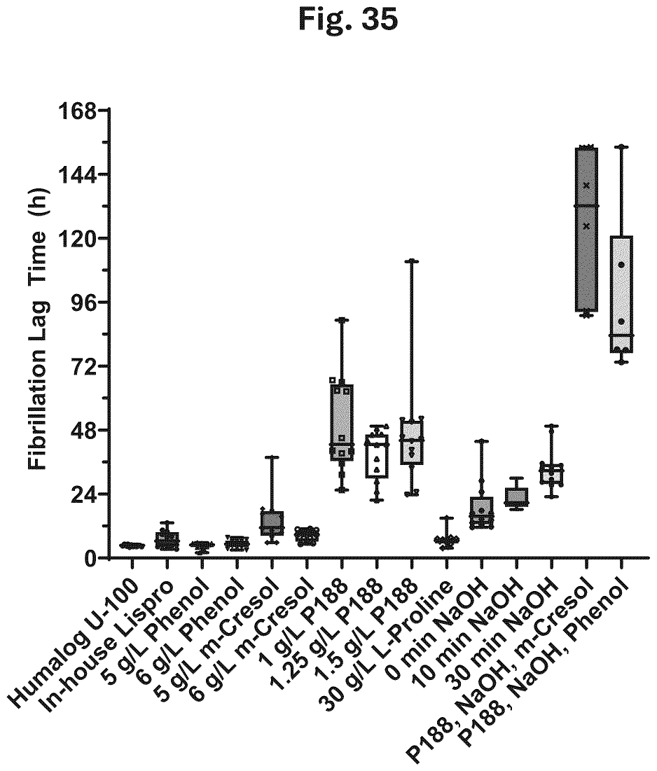

On the nephelometer, commercial Humalog U-100 began fibrillating in just over 4.5 hours on average, with In-house prepared “Humalog” trailing shortly behind at less than 7 hours, as shown in FIG. 35. Replacing phenol with meta-cresol at higher concentrations increased lag time to 8 or 14 hours. Adding L-proline had a lag time of 7 hours, the same as in-house prepared “Humalog.” Poloxamer 188 addition, on the other hand, provided substantial fibrillation protection, reaching a 10-fold improvement above commercial Humalog by experiment's end. Lispro pretreatment with 50 mM NaOH also showed a strong time-dependent increase in lag times compared to control.

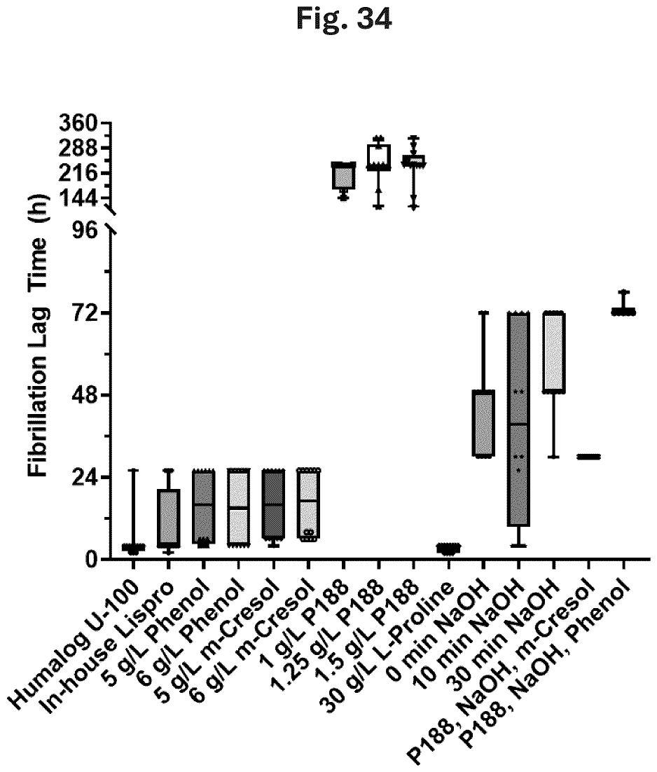

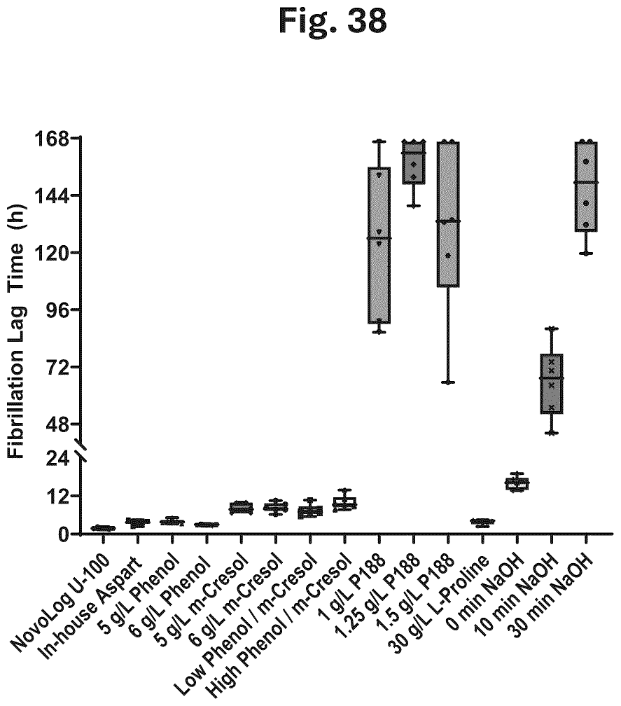

Perhaps even more striking are the synergistic differences when poloxamer 188 and NaOH are combined in either a phenol or meta-cresol background. The presence of phenol seems to have a substantial impact on fibrillation relative to meta-cresol with paired with poloxamer 188 and NaOH pretreatment. While neither of these showed synergistic effects above poloxamer 188 or NaOH pretreatment alone on the rotating mixer, nephelometer conditions led to clear, statistically significant synergies, as is illustrated in FIG. 34. It is noted that in FIG. 34, as well as in each of FIGS. 35, 37, 38, 40 and 41 (all described below), the upper and lower boundaries of boxes respectively indicate first and third quartiles (25th and 75th percentiles), upper and lower whiskers demarcate max and min values, and horizontal lines correspond to median. The vertical positions of the dots represent individual values from the study sample. P values are shown that describe the probabilities that difference between the formulations that are being compared (end points of “boxes” above pairs of formulations) is the result of random error.

The protective effects of poloxamer 188 on lispro seen on the rotating mixer were even greater than those on the nephelometer plate reader, as was the impact of pre-treatment (see FIG. 35), suggesting the singular importance of surfactant in shielding insulin from the liquid-air interface. However, the synergistic effects seen between pre-treatment and poloxamer 188 were not seen on the rotating mixer, perhaps because each of these elements already had a very large effect in this context.

### Example No. 9—Insulin Aspart Formulations

Commercial NovoLog U-100 was acquired and an additional fourteen different formulations of insulin aspart were made. First, eleven different excipient solutions were made by mixing WFI and specified amounts of excipients, one-by-one in the following sequence: WFI, preservative, surfactant (if applicable), tonicity agents, buffers, zinc. The pH of the excipient solution was adjusted to 7.4+/−0.2. In addition to the standard Novolog-mimicking solution (phenol 1.5 mg/mL, meta-cresol 1.72 mg/mL, glycerol at 16 mg/mL, sodium chloride 0.58 mg/mL, dibasic sodium phosphate 1.25 mg/mL, and zinc Q.S. to 0.0196 mg/mL), ten excipient solutions were made in which a single substitution (phenol or meta-cresol at 5 or 6 mg/L plus Phenol+meta-cresol 2.33±2.67 and 2.8±3.2 mg/mL for the meta-cresol/phenol mix, Poloxamer 188 added at 1, 1.25, or 1.5 mg/mL, or L-Proline at 30 mg/mL for the glycerol).

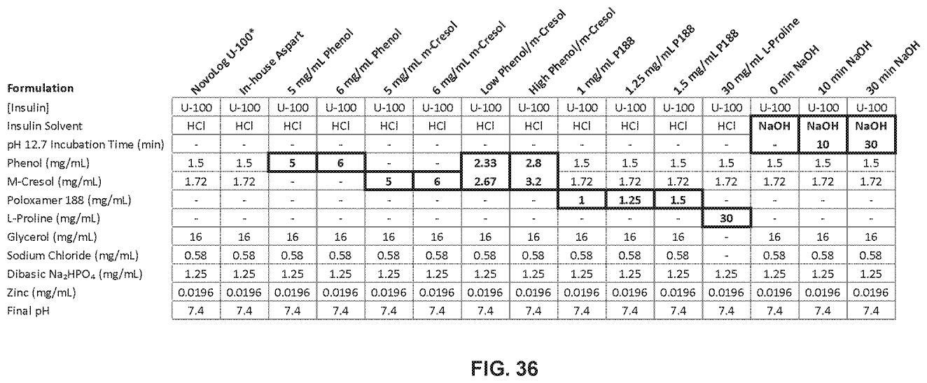

Next, four batches of dissolved aspart were made. One batch was made as follows: appropriate amounts of crystallized insulin aspart to make a U-100 formulation were weighed and mixed with appropriate amounts of acidified WFI; then hydrochloric acid (HCl) was added dropwise while gently mixing for 5-10 minutes until Aspart was fully dissolved without further incubation. Immediately following specified incubation times, the Aspart solution was adjusted to pH 7.4+/−0.2 with NaOH and dissolved into each of the eleven excipient solutions described previously. Three additional batches were then made as follows: appropriate amounts of crystallized insulin aspart to make a U-100 formulation were weighed and mixed with appropriate amounts of 50 mM sodium hydroxide. The insulin aspart was fully dissolved and incubated with gentle mixing for an additional 0, 10, or 30 minutes, respectively. Formulations were then adjusted to pH 7.4+/−0.2 and dissolved into the standard excipient solution. All 14 formulations were then filtered using 0.2-micron syringe filters, and 0.5 mL aliquoted into 2 ml vials. 0.05 mL of commercial Novolog was also alloquoted into a 2 mL vial for use as a control. This is shown in FIG. 36.

These vials were tightly capped and incubated on rotating mixer wheels at 20-60 RPM and 37° C. Turbidity readings were taken every 2-48 hours until samples were fully fibrillated, reaching approximately 1000 nephelometer turbidity units (NTU). Fibrillation lag times were defined as 0.2% of the 1000 NTU formazin standard. Parallel samples were dispensed at 200 μL each into a 96-well microplate and run in a nephelometer plate reader set to 37° C., 700-1000 RPM, and orbital shaking. Readings were captured at 10-15-minute intervals for approximately 3 days or until samples entered the plateau phase of fibrillation. Plate reader fibrillation lag times were defined as 1000 basis units above the upper range of the lag phase baseline, with ascending values continuing into log phase. These lag times were visually verified to denote inflection points of sigmoidal curves.

Fibrillation of rotating mixer and plate reader samples was visually verified for cloudiness. In instances where experiments were stopped before some samples began to fibrillate, lag times are defined as the experimental endpoint.

### Aspart Results

On the nephelometer, commercial NovoLog U-100 began fibrillating in just over 3 hours on average, with In-house prepared Aspart trailing shortly behind at less than 5 hours, shown in FIG. 36. Excipient replacements with varying concentrations of phenol, metacresol, or L-proline failed to significantly extend lag times beyond the NovoLog U-100 control. Poloxamer 188 addition, on the other hand, provided substantial fibrillation protection, reaching a >50-fold improvement above commercial NovoLog by experiment's end. Aspart pretreatment with 50 mM NaOH also showed a strong time-dependent increase in lag times compared to control.

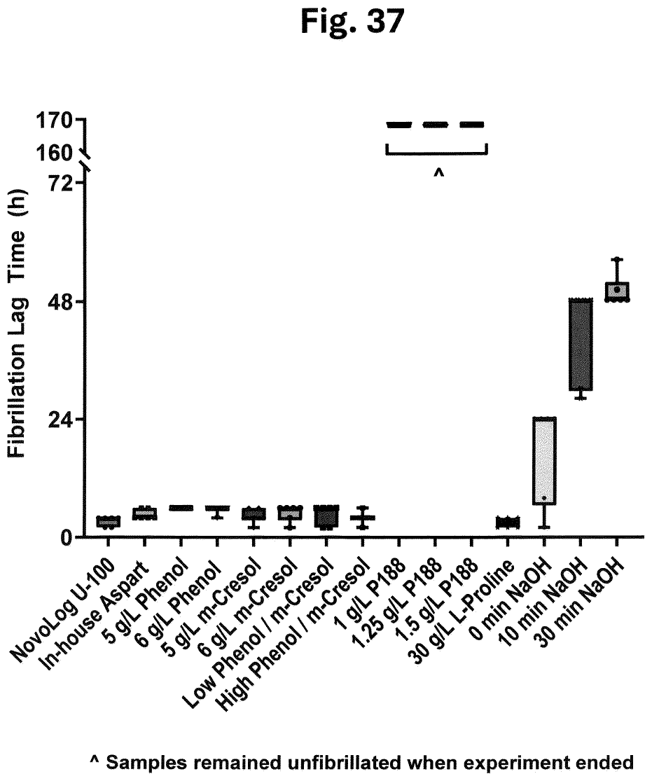

The protective effects of poloxamer 188 and NaOH pretreatment on Aspart seen on the rotating mixer were closely corroborated on the nephelometer plate reader, as shown in FIG. 37. The improved impact poloxamer 188 and reduced effect of NaOH pretreatment data—especially the 30-minute condition, hints at the singular importance of surfactant in shielding insulin from the liquid-air interface.

### Example No. 10—Glargine Formulation

Commercial Lantus U-100 was acquired and an additional twelve different formulations of insulin glargine were made. For the first nine, appropriate amounts of crystallized insulin Glargine to make a U-100 formulation were weighed and mixed with appropriate amounts acidified Water for Injection (WFI), then hydrochloric acid (HCl) was added dropwise while gently mixing until the glargine was fully dissolved. Next were added (one-by-one in the following sequence: preservative, surfactant, tonicity agent, zinc) either the standard components of the Lantus formulation in their standard concentrations (meta-cresol 2.7 mg/mL, polysorbate 20 0.02 mg/mL, glycerol at 17 mg/mL, and zinc 0.03 mg/mL) or the standard components with a single substitution (phenol or meta-cresol at 5 or 6 mg/L for the meta-cresol, Poloxamer 188 at 1, 1.25, or 1.5 mg/mL for the polysorbate, or L-Proline at 30 mg/mL for the glycerol). Formulations were then adjusted to pH 4.0+/−0.2 and filtered using 0.2-micron syringe filters

For the remaining three formulations, appropriate amounts of crystallized insulin Glargine to make a U-100 formulation were weighed and mixed with appropriate amounts of 50 mM sodium hydroxide (NaOH) with subsequent gentle mixing for 0, 10, or 30 minutes. Immediately following specified incubation times, the Glargine solutions were adjusted to pH 4.0+/−0.2 with HCl. Then the Lantus-standard excipients were added one-by-one to their standard concentrations in the following sequence: preservative, surfactant, tonicity agent, stabilizer.

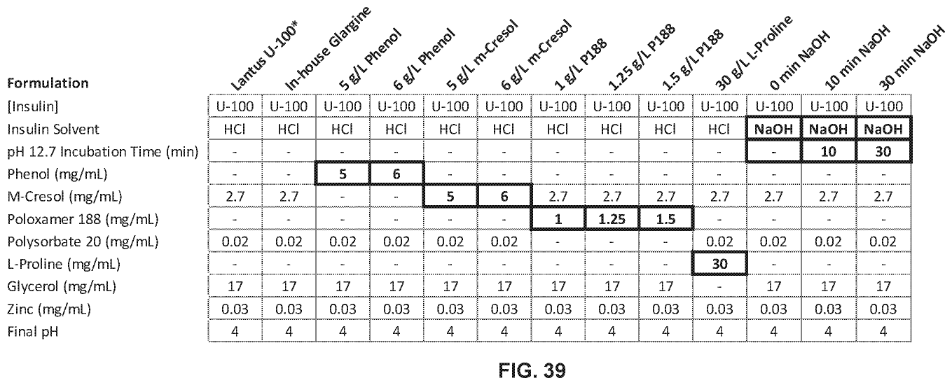

FIG. 39 shows the specific composition of each formulation.

0.5 mL of each formulation (including commercial Lantus U-100) was aliquoted into 2 ml vials. These vials were tightly capped and incubated on rotating mixer wheels at 20-60 RPM and 37° C. Turbidity readings were taken every 2-48 hours until samples were fully fibrillated, reaching approximately 1000 nephelometer turbidity units (NTU). Fibrillation lag times were defined as 0.2% of the 1000 NTU formazin standard.

Parallel samples were dispensed at 200 μL each into a 96-well microplate and run in a nephelometer plate reader set to 37° C., 700-1000 RPM, and orbital shaking. Readings were captured at 10-15-minute intervals for approximately 3 days or until samples entered the plateau phase of fibrillation. Plate reader fibrillation lag times were defined as 1000 basis units above the upper range of the lag phase baseline, with ascending values continuing into log phase. These lag times were visually verified to denote inflection points of sigmoidal curves.

Fibrillation of rotating mixer and plate reader samples was visually verified for cloudiness. In instances where experiments were stopped before some samples began to fibrillate, lag times are defined as the experimental endpoint.

### Results

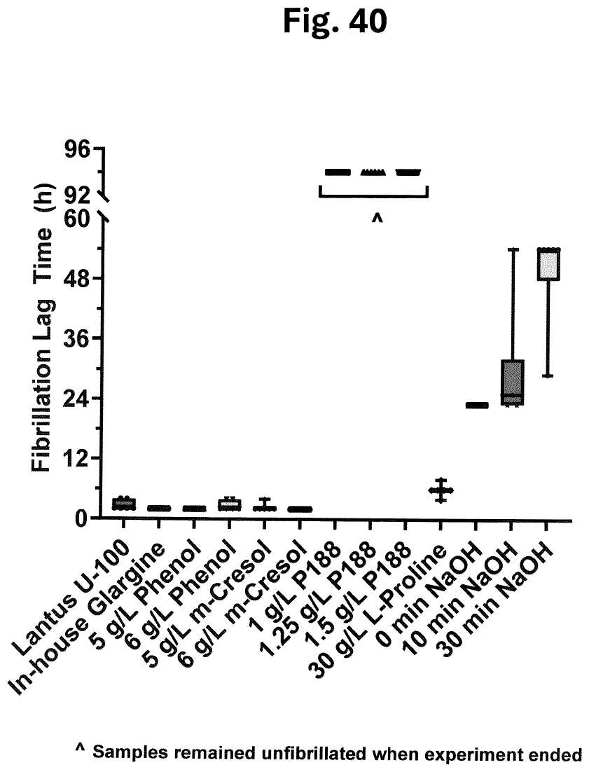

On the rotating mixer wheel, commercial Lantus U-100-similar to the in-house prepared Glargine control-began fibrillating within three hours. Samples varying the concentrations of phenolic preservatives (phenol or metacresol at 5 or 6 mg/mL) saw similar lag times as the control, while L-proline led to a numerical, but non-statistically significant increase in lag time. All poloxamer 188 containing samples, however, significantly decreased fibrillation, showing a >35-fold increase in lag times relative to the Lantus control. Glargine pretreatment in 50 mM NaOH resulted in incubation time-dependent protection of fibrillation, ranging from >8 to >18-fold improvement above the Lantus control. These results are shown in FIG. 40.

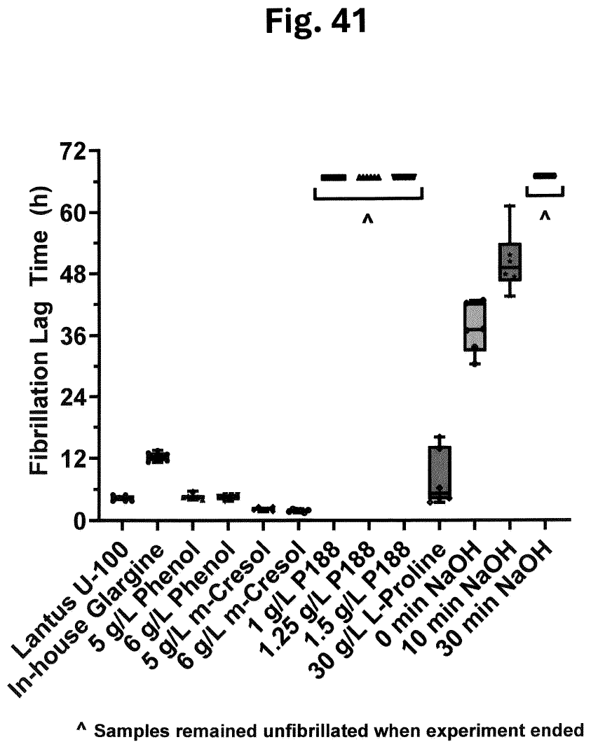

Results for the plate reader largely mirrored the rotating mixer, with poloxamer 188 and 50 mM NaOH pretreatment significantly extending lag times, are shown in FIG. 41. Unsurprisingly, the impact on nephelometer lag times is less pronounced for poloxamer 188 than on the rotating mixer wheel: the wheel repeatedly inverts the samples so that the liquid-air interface—and the utility of a surfactant—is maximized, whereas the orbital mixing of the plate reader produced minimal liquid-air interfaces, even at high RPM. The rotating mixer is likely a more realistic albeit accelerated model of stress an insulin formulation is likely to experience in the real world. Although particular embodiments, aspects, and features have been described and illustrated, it should be noted that the invention described herein is not limited to only those embodiments, aspects, and features. It should readily be appreciated that modifications may be made by persons skilled in the art. The present application contemplates any and all modifications within the spirit and scope of the underlying embodiments described and claimed herein, and all such embodiments are within the scope and spirit of the present disclosure.

### LIST OF REFERENCES

1. Bhutta Z A, Salam R A, Gomber A, Lewis-Watts L, Narang T, Mbanya J C, Alleyne G. A century past the discovery of insulin: global progress and challenges for type 1 diabetes among children and adolescents in low-income and middle-income countries. Lancet. 2021 Nov. 13; 398 (10313): 1837-1850. doi: 10.1016/S0140-6736 (21) 02247-9. PMID: 34774146.
2. Brange, J., Andersen, L., Laursen, E. D., Meyn, G., & Rasmussen, E. (1997). Toward understanding insulin fibrillation. J Pharm Sci, 86 (5), 517-525. https://doi.org/10.1021/js960297s
3. Brange, J., & Langkjoer, L. (1993). Insulin structure and stability. Pharm Biotechnol, 5, 315-350. https://doi.org/10.1007/978-1-4899-1236-7_11
4. Devi, S. (2021). Cold comfort: getting insulin to those who need it. Lancet, 398 (10313), 1791. https://doi.org/10.1016/S0140-6736 (21) 02482-X
5. Heinemann, L., Braune, K., Carter, A., Zayani, A., & Kramer, L. A. (2021). Insulin Storage: A Critical Reappraisal. J Diabetes Sci Technol, 15 (1), 147-159. https://doi.org/10.1177/1932296819900258
6. Heise, T., Meiffren, G., Alluis, B., Seroussi, C., Ranson, A., Arrubla, J., Correia, J., Gaudier, M., Soula, O., Soula, R., DeVries, J. H., Klein, O., & Bode, B. (2019). BioChaperone Lispro versus faster aspart and insulin aspart in patients with type 1 diabetes using continuous subcutaneous insulin infusion: A randomized euglycemic clamp study. Diabetes Obes Metab, 21 (4), 1066-1070. https://doi.org/10.1111/dom. 13621
7. Huus, K., Havelund, S., Olsen, H. B., van de Weert, M., & Frokjaer, S. (2006). Chemical and thermal stability of insulin: effects of zinc and ligand binding to the insulin zinc-hexamer. Pharm Res, 23 (11), 2611-2620. https://doi.org/10.1007/s11095-006-9098-y
8. Jarosinski, M. A., Chen, Y. S., Varas, N., Dhayalan, B., Chatterjee, D., & Weiss, M. A. (2022). New Horizons: Next-Generation Insulin Analogues: Structural Principles and Clinical Goals. J Clin Endocrinol Metab, 107 (4), 909-928. https://doi.org/10.1210/clinem/dgab849
9. Jarosinski, M. A., Dhayalan, B., Chen, Y. S., Chatterjee, D., Varas, N., & Weiss, M. A. (2021). Structural principles of insulin formulation and analog design: A century of innovation. Mo/Metab, 52, 101325. https://doi.org/10.1016/j.molmet.2021.101325
10. Mann, J. L., Maikawa, C. L., Smith, A. A. A., Grosskopf, A. K., Baker, S. W., Roth, G. A., Meis, C. M., Gale, E. C., Liang, C. S., Correa, S., Chan, D., Stapleton, L. M., Yu, C., Muir, B., Howard, S., Postma, A., & Appel, E. A. (2020). An ultrafast insulin formulation enabled by high-throughput screening of engineered polymeric excipients. Science Translational Medicine, 12 (550), DOI: 10.1126/scitranslmed.abd6726. https://doi.org/10.1126/scitranslmed.aba6676
11. Pepelnjak, M., Velten, B., Napflin, N., van Rosen, T., Palmiero, U. C., Ko, J. H., Maynard, H. D., Arosio, P., Weber-Ban, E., de Souza, N., Huber, W., & Picotti, P. (2024). In situ analysis of osmolyte mechanisms of proteome thermal
12. stabilization. Nat Chem Biol, 20 (8), 1053-1065. https://doi.org/10.1038/s41589-024-01568-7
13. Qafary, M., Rashno, F., Khajeh, K., Khaledi, M., & Moosavi-Movahedi, A A (2022). Insulin fibrillation: Strategies for inhibition. Prag Biophys Mo/Biol, 175, 49-62. https://doi.org/10.1016/j.pbiomolbio.2022.09.001
14. Webber, M. J., Appel, E. A, Vinciguerra, B., Cortinas, A B., Thapa, L. S., Jhunjhunwala, S., Isaacs, L., Langer, R., & Anderson, D. G. (2016). Supramolecular PEGylation of biopharmaceuticals. Proc Natl Acad Sci USA, 113 (50), 14189-14194. https://doi.org/10.1073/pnas. 1616639113
15. Woods, R. J., Alarcon, J., McVey, E., & Pettis, R. J. (2012). Intrinsic fibrillation of fast-acting insulin analogs. J Diabetes Sci Technol, 6 (2), 265-276. https://doi.org/10.1177/193229681200600209

1. A pharmaceutical formulation, comprising:
an insulin or an insulin analog exposed to a pH in excess of 8 for at least 20 minutes; and
at least one protective excipient, in high concentration, from the following: a protective osmolyte, a surfactant, and a phenolic preservative.

2. The pharmaceutical formulation of claim 1, wherein the insulin or insulin analog is one or more of: regular human insulin, insulin lispro, insulin aspart, insulin glulisine, insulin glargine, insulin detemir, or insulin degludec.

3. The pharmaceutical formulation of claim 2, wherein at least one of:
the concentration of the protective osmolyte confers an osmolarity greater than 250 mOsm/L;
the concentration of the surfactant is greater than 0.1% w/v; or
the concentration of the phenolic preservative is greater than 0.40% w/v.

4. The pharmaceutical formulation of claim 1, wherein the concentration of the surfactant is below the critical micelle concentration.

5. The pharmaceutical formulation of claim 1, wherein the insulin or insulin analog is first exposed to the pH in excess of 8 for no more than 40 minutes, and then the one or more excipients are added to the formulation.

6. The pharmaceutical formulation of claim 1, wherein at least one of: the formulation is free of added chloride ions, the insulin or insulin analog is first exposed to the pH in excess of 8 for 30 minutes, and the protective osmolyte is L-Proline.

7. The pharmaceutical formulation of claim 1, wherein the concentration of the insulin or insulin analog is greater than 100 IU/ml.

8. The pharmaceutical formulation of claim 1, wherein the formulation is free of glycerol or other non-protective osmotic agents.

9. The pharmaceutical formulation of claim 3, wherein the concentration of phenolic preservative is between 0.50% and 0.60% w/v.

10. The pharmaceutical formulation of claim 1, wherein the phenolic preservative is one of: phenol, metacresol, or a combination of both.

11. The pharmaceutical formulation of claim 1, wherein the phenolic preservative is a paraben, phenoxyethanol, benzyl alcohol, or any combination thereof.

12. A pharmaceutical formulation, comprising:
human insulin at 500 IU/ml;
a combination of phenol and metacreasol at a concentration of between 0.40 and 0.75% w/V;
poloxamer 188 at a concentration of between 1.5 and 1.8 mg/ml;
L-Proline at a concentration of between 25 and 35 mg/ml; and
zinc sulfate at a concentration of between 0.08 and 0.1 mg/ml.

13. The pharmaceutical formulation of claim 9, wherein the formulation is free of chloride ions.

14. The pharmaceutical formulation of claim 9, wherein the insulin or insulin analog is first solubilized in 50 mM sodium hydroxide (NaOH) with subsequent mixing for between 20 and 40 minutes, and then adjusting its pH to from 7.0-7.8.

15. The pharmaceutical formulation of claim 11, wherein the pH is adjusted utilizing sulfuric acid.

16-19. (canceled)

20. A method of preparing a pharmaceutical formulation, comprising:
solubilizing an insulin or insulin analog in 50 mM sodium hydroxide (NaOH);
mixing the solution between 20-40 minutes;
lowering a pH of the solution to between 7.0-7.8 using an acid; and
adding at least one protective excipient from among a protective osmolyte, a surfactant, and a phenolic preservative.

21. The method of claim 20, wherein the wherein the pH is adjusted utilizing sulfuric acid.

22-26. (canceled)

27. The method of claim 20, wherein the pharmaceutical formulation is free of chloride ions.

28. The method of claim 20, further comprising delivering the pharmaceutical formulation at least one of: transdermal, subcutaneously, intravenously, nasally, buccally, or into the intraperitoneal space.

29. The method of claim 20, further comprising delivering the pharmaceutical formulation via at least one of: syringes, pens, patches, inhalers, or pumps.
